(19) United States
(12) Patent Application Publication
(10) Pub. No.: US 2003/0092064 A1
(43) Pub. Date: May 15, 2003

(54) System for the synthesis of spatially separated libraries of compounds and methods for the use thereof

(75) Inventor: John C. Reader, Cambridge (GB)

Correspondence Address: Dike, Bronstein, Roberts & Cushman, Intellectual Property Pratice Group, Edwards & Angell, LLP, P.O. Box 9169, Boston, MA, 02209, US

(73) Assignee: Millennium Pharmaceuticals, Inc.

(21) Appl. No.: 10/116,860

(22) Filed: Apr. 5, 2002

Related U.S. Application Data
(60) Provisional application No. 60/281,657, filed on Apr. 5, 2001.

(51) Int. Cl. G01N 33/53; G01N 33/543

(52) U.S. Cl. 435/7.1; 436/518

(57) ABSTRACT

Featured is an apparatus useful for preparing combinatorial libraries of compounds in a parallel fashion so that the individual compounds of the library are spatially separate and the position of each compound of the library in the apparatus is known. Moreover, the apparatus is useful for the preparation of combinatorial libraries of compounds where the library is constructed from three or more building blocks, e.g., three, four, five, six or seven building blocks, resulting in a three, four, five, six or seven dimensional combinatorial library. Also featured are related methods for the preparation of three, four, five, six or seven dimensional combinatorial libraries of compounds using the apparatus of the present invention.

This application claims the benefit of U.S. Provisional Application Serial No. 60/281,657 filed Apr. 5, 2001, the teachings of which are incorporated herein by reference.

### FIELD OF INVENTION

The present invention relates to a system for the synthesis of a spatially defined multi dimensional library of chemical compounds where each compound can be identified in the library by referring to sufficient coordinates to define a specific location in the multi dimensional space. More particularly the present invention relates to a 3, 4, 5, 6 or 7 dimensional solid phase combinatorial library of chemical compounds wherein the synthetic history of each of the solid supports in the library is identifiable based on 3, 4, 5, 6 or 7 coordinates and methods of generating these combinatorial libraries.

### BACKGROUND OF THE INVENTION

Large collections (libraries) of molecules have emerged as important tools for the successful identification of useful compounds. Such libraries have typically been synthesized using combinatorial approaches (see, e.g., Gallop, et al., 1994; Gordon, E. M., et al., 1994). Several different methods have been used to assemble combinatorial libraries of various compounds. One such methodology for peptide or oligonucleotide synthesis was developed by Affymax Technologies N.V. and disclosed in U.S. Pat. No. 5,143,854. The Affymax method involves sequentially using light for illuminating a plurality of polymer sequences on a substrate to expose reactive functional groups and delivering reaction fluids to said substrate. This method of synthesis produces large numbers, but relatively small quantities of products. A further method and device for producing peptides or oligonucleotides is disclosed in Houghton, E. P. O. 196174. Houghton's apparatus is a polypropylene mesh container or sac, similar to a tea-bag, which encloses reactive particles.

While combinatorial chemistry synthetic schemes such as the methods described above can generate large numbers of different compounds with a minimum number of steps, they have certain disadvantages. As mentioned above, some of the methods are capable of producing only limited quantities of each compound. Furthermore, the compounds are often synthesized and screened in “pools” or “batches”. This can result in loss of potentially valuable information during screening if, for example, a particular pool contains compounds that possess agonist activity and compounds with antagonist activities. Further, once a pool is identified as containing a potentially active compound, the identity of the active compound must be determined. This identification or decoding requires some type of deconvolution or tagging protocol, requiring additional steps to identify the active compound.

Parallel synthesis strategies do not suffer from the above-mentioned disadvantages of combinatorial approaches, as a single compound is generated and assayed (see, e.g., Sugarman, et al., U.S. Pat. No. 5,503,805, issued Apr. 2, 1996). The disadvantage of parallel synthesis strategies is that presently-available automated instrumentation for carrying out such syntheses is costly and complex, requiring large numbers of valves, separate pieces of tubing, and the like. Accordingly, it is generally not suitable for the synthesis of large numbers (e.g., >100) of compounds. Currently available automated parallel synthesis instruments are typically limited in their capacity to between 12 and 96 reaction vessels. Using manual instruments or reaction blocks is less costly but throughput is reduced and typical format is just 96 reaction vessels.

The three dimensional combinatorial library system disclosed by Campbell et al. in U.S. Pat. No. 6,083,682 involves a plurality of middle plates which receive interleaving membranes with a two dimensional array of holes in the x, y plane to form a three dimensional array having x, y, and z axes defined by Z (x, y) reaction planes that are different sheets of membrane. The membranes are stacked between a pair of end plates that have such plumbing to control the delivery of fluids to reaction zones with common z coordinate. The device is used for the parallel synthesis of compounds on to one or more membranes.

Fluid delivery in Campbell is accomplished by pressurizing the fluid before introduction into the 3-D array. Each reaction zone has to be isolated in a (x, y) plane to prevent fluid leakage and contamination of the array. The number of plates stacked in the z coordinate is limited by the antagonistic relation between increasing fluid delivery pressure to increase fluid flow rate and increasing likelihood of contamination between reaction zones with a common z coordinate and different (x, y) coordinates.

The combinatorial synthesis array system disclosed by Campbell requires that each reaction membrane be sandwiched between divider plates. Compression of the 3-D assembly results in the isolation of each reaction zone by compression of the area surrounding each reaction zone. The end plates of the array comprise a complicated fluid delivery apparatus that is able to selectively direct individual fluid mixtures to any of the Z(x, y) columns of reaction zones. The fluids are pressurized to drive the fluid through the stacked membranes so that the end plates and the membrane seals must be able to withstand the required pressures. Additionally there are a large number of separate fluid reservoirs and fluid pressurizing assemblies to deliver individual fluid mixtures to each Z(x, y) column of reaction zones.

It thus would be desirable to provide new methods suitable for the preparation of a solid-phase combinatorial library of chemical compounds where the synthetic history of each solid support of the library is known based on the location of the support in the device. Moreover, it would be desirable to provide an apparatus for use with such methods where the devices do not require an extensive amount of specialized equipment and are highly adaptable for application in preparing a variety of libraries of chemical compounds.

### SUMMARY OF THE INVENTION

The invention provides, in one aspect, a method and apparatus for synthesizing chemicals onto solid supports in a combinatorial manner. The apparatus includes a plurality of reaction columns, each of which comprises at least two reaction zones. The reaction zones, each of which comprises at least one solid support, are arranged in a three-dimensional reaction block, e.g. an array of reaction zones that have x, y, and z axes defining individual reaction zones. In such reaction blocks, reaction zones having common (x, y) coordinates and different z coordinates form a vertical stack of reaction zones, e.g., a “column” of reaction zones. Similarly, reaction zones having common z coordinates but different (x, y) coordinates form a two-dimensional “reaction plane” of reaction zones. In systems with one reaction block, a three-dimensional combinatorial library of compounds can be prepared. A four-dimensional library of compounds can be prepared using a system of the invention that has a plurality of reaction blocks arranged in a one dimensional array, e.g., a linear array that has an x′ axis, such that individual reaction zones are identified by four coordinates (x, y, z, x′). A five-dimensional combinatorial library of compounds can be prepared using a system of the invention that has a plurality of reaction blocks arranged in a two-dimensional array, e.g., a square or rectangular array, having x′ and y′ axes. Individual reaction zones of a five-dimensional system are defined by five coordinates (x, y, z, x′, y′). A six-dimensional combinatorial library of compounds can be prepared using a system of the invention that has a plurality of reaction blocks arranged by “stacking” two or more square or rectangular arrays of reaction blocks to form a three-dimensional array of reaction blocks, e.g., a cube or rectangular prism, having x′, y′ and z′ axes. Individual reaction zones of a six-dimensional system are defined by six coordinates (x, y, z, x′, y′, z′).

In other preferred embodiments, a 4, 5 or 6 dimensional library where each reaction zone of the library is uniquely identified by 4, 5 or 6 coordinates can be prepared in one or more composite reaction blocks. Each composite reaction block has at least one composite axis that is dependent upon two coordinates of the library, e.g., the coordinate values for x and x′, y and y′, and/or z and z′ are combined to form a composite coordinate. For example, values of composite coordinates a, b and c are determined from the values of x and x′, e.g., a=X(x′−1)+x, y and y′, e.g., b=Y(y′−1)+y, or z and z′, e.g., c=Z(z′−1)+z, where X, Y, and Z are the number of different compositions for each diversity introducing reaction step, e.g., a reaction or process that introduces diversity into the library of compounds, which is varied along the x, y and z axis of the library. A composite reaction block preferably comprises all (x, y, z) reaction zones that will receive different compositions for a diversity introducing reaction step that introduces diversity into the library of compounds according to the one or more of the x′, y′, z′ coordinates of the multidimensional axis of the library.

Preferably the library can be prepared in one composite reaction block that comprises all the reaction zones of the assembly. Further the composite reaction block has the reaction zones arranged in a rectangular prismatic array with axes defining three composite coordinates for a six dimensional library, e.g., a, b and c wherein:

a=X(x′−1)+x

b=Y(y′−1)+y

c=Z(z′−1)+z′

Using the a, b and c composite coordinates defined above results in a composite reaction block wherein there are regions of the block with reaction zones with reaction histories that have different x, y and z coordinates and common x′, y′, and z′ coordinates. Clearly, the organization of the reaction block is arbitrary and any other arrangement of reaction zones into regions with different common coordinates are also suitable for use with the present invention.

Two or more reaction blocks of a four, five or six-dimensional array can be combined to form larger reaction blocks to facilitate the addition of appropriate reagents and building blocks to each reaction zone of the library array. Alternatively, the dimensions of a single reaction block can be extended in the x, y and z dimensions depending on the number of variables, e.g., reactions that introduce diversity into the library, within the reaction steps that introduce diversity.

For example, in a method for a six-dimensional library of compounds, reaction blocks with common (x′, y′) coordinates can be combined in the z′ direction so that the reaction zones with the same x′ and y′ coordinates are stacked together to form a two-dimensional, e.g., a square or rectangular, array of larger reaction blocks. Each larger reaction block then receives the appropriate fourth and fifth building block compositions. The final building block, e.g. the building block that is varied along the z′ axes, can be introduced after the larger reaction blocks are split into separate reaction blocks.

In a similar example for a five-dimensional library of compounds, reaction blocks with a common x′ coordinate can be combined in so that reaction blocks with the same x′ coordinate but different y′ coordinate are vertically stacked. The building block that is varied along the x′ axis is then contacted with the appropriate building block. The larger reaction blocks are then disassembled and the fifth building block introduced in to the reaction blocks with the corresponding y′ coordinate value.

A six dimensional array of reaction zones can be arranged in a two-dimensional array of composite reaction block where each reaction block comprises a three dimensional array of reaction zones where each reaction zone is uniquely identified by its (x, y, c) coordinates. The c composite coordinates is dependent upon two coordinates of the six coordinates along which diversity introducing reaction steps of the library are varied, e.g., c=Z(z′−1)+z where Z is the number of different chemical compositions of the diversity introducing reaction step that is varied along the z coordinate of the six dimensional library of chemical compounds.

The reaction columns of the reaction block are arranged in a two-dimensional array. Reaction columns can have a solid bottom, e.g., a test tube. Alternatively, reaction columns can have an open bottom that allows liquids and gases to pass through the column but prevents the solid supports contained in the reaction column to exit the column. Preferred openings include holes,sintered frits,meshes,bars or the like that optionally include a reversibly closeable valve that can create a sealed bottom so that liquids and gases cannot exit the bottom of the reaction column while the valve is in the closed position. For reaction blocks that have reaction columns with optionally closeable valves disposed therein the reaction column can be opened or closed in concert with some or all of the other reaction column valves of the reaction block or the valve can be opened or closed in an isolated event that does not effect the other reaction column valves of the reaction block.

Exemplary materials suitable for use as solid supports with the present invention include lanterns™, beads, CD plugs (B. Atrash, M. Bradley, R. Kobylecki, D. Cowell, J. Reader, Angewandte Chemie, (2001) 113, pp. 964-967), Crowns™, Irori kans™, paper discs, functionalized polymer discs, rods, tubes or polyhedra.

Exemplary reaction blocks suitable for use with the present invention include standard 96 well (8×12) multiwell plates that are compatible with Robbins' Block, Bohdan miniblocks, Radley's Combiclamp, and like components and Robbins Flexchem™ reaction block that can receive two or more solid supports in each well of the reaction block such that the dimensions of the reaction column relative to the solid-supports does not allow the supports to pass each other in the z-axis. Further, the relation between the dimensions of the reaction column and the solid support is such that liquids and gases that are introduced in to the reaction column are able to pass or permeate through the solid supports of the reaction column.

In one aspect, the invention provides a method of synthesizing a library of compounds in a reaction block that includes a two dimensional array of reaction columns. Each reaction column can comprise at least two reaction zones and the solid support(s) of an individual reaction zone generally cannot exchange position with adjacent solid supports of other reaction zones in the reaction column. The method includes the steps of (i) derivatizing batches of solid supports with a different chemical composition in a first diversity introducing reaction step such that different batches have a different first diversity introducing reaction history; (ii) charging a reaction block of reaction columns with solid supports with the same reaction history for the first diversity introducing reaction step so that the solid supports with a common reaction history form a reaction plane in an (x, y) plane, e.g, the solid supports are located in reaction zones with a common z coordinate but different (x, y) coordinates; (iii) charging the reaction blocks with solid supports with different reaction history of the first diversity introducing reaction step to form a series of parallel (x, y) reaction planes wherein each reaction plane comprises reaction zones and solid supports with a common reaction history for the first diversity introducing reaction step and the solid support(s) from one reaction zone generally cannot exchange location with a solid support from an adjacent reaction zone, e.g., a reaction zone with the same (x, y) coordinates and a z coordinate differing by 1 position; (iv) delivering chemical composition to reaction zones having a common x coordinate value such that they receive the same chemical composition for the second diversity introducing reaction step and reaction zones that have different x coordinate values generally receive different chemical compositions for the second diversity introducing reaction step; and (v) delivering chemical composition to reaction zones having a common y coordinate value such that they receive the same chemical composition for the third diversity introducing reaction step and reaction zones that have different y coordinate values generally receive different chemical compositions for the third diversity introducing reaction step. Each reaction zone of the reaction block received a different combination of chemical compositions for the first, second and third diversity introducing reaction steps so that each reaction zone has a different reaction history uniquely identified by the corresponding (x, y, z) coordinate value.

There is no particularly preferred correlation between a specified diversity introducing reaction step and a given axis in the reaction assembly. For example in the above-described method, the first diversity introducing reaction step was varied along the z-axis, the second diversity introducing reaction step was varied along the x-axis, and the third diversity introducing reaction step was varied along the y-axis. However any other method of introducing the chemical compositions of the diversity introducing reaction steps by changing the sequence of axis along which each reaction diversity step is introduced would also be acceptable, e.g., for example, a method wherein the first diversity introducing reaction step was varied along the x-axis, the second diversity introducing reaction step was varied along the y-axis, and the third diversity introducing reaction step was varied along the z-axis.

In another aspect, the invention provides a method of synthesizing a library of compounds. The method includes the steps of (i) derivatizing batches of solid supports with a different first building block composition; (ii) distributing solid supports with a common first building block to the wells of a reaction plane such that there is at least one solid support per reaction plane well; (iii) preparing two or more reaction planes in the first building block to form a three-dimensional array of discrete reaction zones and a two-dimensional array of reaction columns corresponding to the array of solid supports, where each reaction zone contains at least one solid support and where each reaction column contains at least two reaction zones such that each reaction zone in a reaction column has common (x, y) coordinates but different z coordinates; (iv) delivering a second building block to the reaction zones such that zones having a common x coordinate value receive the same second building block; and (v) delivering a third building block to the reaction zones such that zones having a common y coordinate value are contacted with the same third building block. The reaction of the second and third building blocks in the different reaction zones of the three-dimensional array thus forms the library of compounds. The library of compounds is formed by the reaction of the first, second and third building blocks in the different reaction zones. The solid supports may be any solid support suitable for performing chemical syntheses, as described above.

The reaction planes may be arranged to form a stack, flanked by one or two end plates as necessary to prevent reagent loss from the reaction block. For example a bottom end plate may be attached so that liquid reagents or solutions can be added to the reaction block. Top and bottom end plates may be attached so that a liquid filled reaction block can be effectively agitated or a reaction block can be pressurized with a gas.

In another aspect, the invention provides a method of synthesizing a library of compounds via a sequence of 4 or more reaction steps at which diverse synthon compositions, e.g. diversity introducing reaction steps, can be introduced selectively to specific reaction zones of the reaction assembly such that all compounds resulting from all building block combinations are spatially separate and addressable with 4 or more coordinates. The method for the preparation of a combinatorial library of compounds with six reaction steps at which diversity can be introduced, e.g., diversity introducing reaction steps, includes the steps of (i) preparing a plurality of reaction blocks that have the same compounds located in equivalent reaction zones, e.g., a plurality of equivalent three dimensional libraries of compounds prepared by the method above described; (ii) the plurality of reaction zones are arranged in a cubic or rectangular prismatic array having coordinates x′, y′ and z′; (iii) delivering a fourth building block to the reaction zones such that zones having a common z′ coordinate value receive the same fourth building block; (iv) delivering a fifth building block to the reaction zones such that zones having a common y′ coordinate value receive the same fifth building block; and (v) delivering a sixth building block to the reaction zones such that zones having a common x′ coordinate value are contacted with the same sixth building block.

A library of compounds generated by the combination of five sets of synthons in five diversity introducing reaction steps can be prepared by the above described method for preparing a six-dimensional library of compounds. Step (ii) is modified such that the plurality of reaction blocks are arranged in a two-dimensional array, e.g., a square or rectangle, having x′ and y′ axes. Each reaction block of the array of reaction blocks has z′=1 so each reaction zone of the array can be addressed by five coordinates (x, y, z, x′, y′) where the variable z′ is a constant in the five-dimensional array and have been omitted for clarity. Further, step (iii) of the method for the preparation of a six-dimensional library can be omitted because z′=1 for all of the reaction zones of the library, e.g., a five-dimensional library will typically only have five reaction steps wherein diversity is introduced into the library of chemical compounds.

A library of chemical compounds generated by the combination of four sets of synthons in four diversity introducing reaction steps can be prepared according to the above described method for preparing a six-dimensional library of compounds. Step (ii) is modified such that the plurality of reaction blocks are arranged in a one-dimensional array, e.g., a line, having an x′ axis. Each reaction block of the array of reaction blocks has y′=1 and z′=1 so each reaction zone of the array can be addressed by four coordinates (x, y, z, x′) where the variables y′ and z′ are constant in the four-dimensional array and have been omitted for clarity. Further, steps (iii) and (iv) of the method for the preparation of a six-dimensional library can be omitted because (y′, z′)=(1, 1) for all of the reaction zones of the library, e.g., a four-dimensional library will typically only have four diversity introducing reaction steps wherein diversity is introduced into the library of chemical compounds.

The invention also provides another exemplary method for preparing a three-dimensional combinatorial library of compounds. According to the method, a plurality of reaction zones is provided where the reaction zones are arranged in a three dimensional array such that each reaction zone is identifiable with a unique set of (x, y, z) coordinates. The number of reaction zones is preferably represented as (X Y Z), which notation represents the product of X, Y, and Z, where X, Y, and Z represent integers. For example, if X=2, Y=3 and Z=4, (X Y Z) would be equal to 24. The reaction zones are preferably arranged in a three-dimensional array having x, y and z axes. Accordingly, if the same numbers are used, the array of 24 reaction zones has the dimensions of 2 zones along the x-axis, 3 zones along the y-axis, and 4 zones along the z-axis. The location of each reaction zone in the array is defined by its (x, y, z) coordinates in the array, e.g., a particular zone may have the coordinates (1, 3, 2). It follows that 2-dimensional planes or arrays of zones may be defined by holding one of the coordinate values constant, e.g., a (y, z) reaction plane of reaction zones is defined by a common x coordinate value.

Similarly, for libraries of chemical compounds prepared using four, five or six reactions that introduce diversity into the array, a suitable reaction assembly will include a one, two or three dimensional array of reaction blocks. According to the method, a plurality of reaction zones is provided. The number of reaction zones is preferably represented as (XYZX′Y′Z′), which notation represents the product of X, Y, Z, X′, Y′ and Z′, where X, Y, Z, X′, Y′ and Z′ are integers corresponding to the number of permutations in the diversity introducing reaction steps that is varied in the corresponding axis, e.g., X chemical compositions for the diversity introducing reaction step that varies along the x-axis, and so on for the other axes. For example, if X=2, Y=3, Z=4 X′=2, Y′=3, and Z′=4, (XYZX′Y′Z′) would be equal to 576. The reaction blocks are preferably arranged in a three dimensional array of reaction blocks having x′, y′ and z′ axes and reaction zones are preferably arranged in a three dimensional array of reaction zones having x, y and z axes in each reaction block. In particularly preferred reaction assemblies, the reaction blocks are arranged in a two dimensional array of reaction blocks having x′ and y′ axes and reaction zones are preferably arranged in a two dimensional array of reaction zones having x, y and z axes in each reaction block. Accordingly, if the same numbers are used, the array of 576 reaction zones has the dimensions of 2 reaction zones along the x-axis, 3 reaction zones along the y-axis, 4 reaction zones along the z-axis, 2 reaction zones along the x′-axis, 3 reaction zones along the y′-axis, and 4 reaction zones along the z′-axis. The location of each reaction zone in the array is defined by its (x, y, z, x′, y′, z′) coordinates in the array, e.g., a specified reaction zone may have the coordinates (1, 3, 2, 1, 2, 1). It follows that 2-dimensional planes or arrays of reaction zones or reaction blocks may be defined by holding one of the coordinate values constant, e.g., a (y, z) reaction plane of reaction zones is defined by a common x coordinate and refers to all (y, z) reaction planes in each reaction block of the array that has a common x coordinate. Similarly a (y′, z′) reaction block plane of reaction blocks is defined by a common x′ coordinate and refers to all reaction zones with all (x, y, z) coordinate combinations contained within the (y′, z′) reaction blocks with a common x′ coordinate.

The invention provides another exemplary method for preparing a combinatorial library of compounds. According to the method, a plurality of reaction zones, each containing at least one solid support scaffold, is provided. The number of reaction zones is preferably represented as (XYZX′Y′Z), the product of X, Y, Z, X′, Y′ and Z′, where X, Y, Z, X′, Y′ and Z′ represent positive integers. For example if X=2, Y=3, Z=4, X′=5, Y′=6 and Z′=7 then XYZX′Y′Z is equal to 5040. The wells or reaction zones are arranged in a two-dimensional array on reaction plates having x and y-axes. A plurality of reaction plates are stacked vertically to form a reaction block that has wells arranged in a three-dimensional array having x, y and z axes. Accordingly, if the same numbers are used, each reaction block includes 24 wells or reaction zones, e.g. X Y Z or 2×3×4, having the dimensions of 2 wells along the x axis, 3 wells along the y axis and 4 reaction zones along the z axis. The reaction blocks are organized in a three dimensional array having x′, y′ and z′ axes such that each reaction block of X Y Z (24) reaction zones is defined by its (x′, y′, z′) coordinates. Accordingly, if the same numbers are still used, the reaction assembly includes 210 reaction blocks, e.g. X′ Y′ Z′ or 5×6×7, having dimensions of 5 reaction blocks along the x′ axis, 6 blocks along the y′ axis and 7 blocks along the z′ axis. The location of each reaction zone in the reaction assembly is defined by its six coordinates (x, y, z, x′, y′, z′), e.g., a particular well having the coordinates (1, 3, 2, 1, 4, 6). It follows that library subsets may be defined by holding one or more of the coordinate values constant. For example, (x, y, z, 2, 3, 4) defines a subset of reaction zones that comprises all the reaction zones of the reaction block located at (x′, y′, z′)=(2, 3, 4) and (1, 2, 3, x′, y′, z′) defines a subset of reaction zones that comprises each reaction with (x, y, z)=(1, 2, 3) located in each reaction block of the array of reaction blocks.

In certain preferred embodiments, the location of a reaction zone in the reaction assembly which is defined by its six coordinates (x, y, z, x′ y′ z′) can be arrayed in a reaction assembly with a two dimensional array of reaction blocks where two coordinates defining the reaction history of a reaction zone are preferably varied in one axis called a composite coordinate or composite axis. In an illustrative example, a w axis is defined as w=Y′(z′−1)+y′ wherein the diversity introducing reaction steps varied along the y′ and z′ coordinates are introduced into selected reaction zones along the w composite axis.

The term “diversity introducing reaction step” refers to a reaction or series of reactions wherein one or more chemical compositions are added to specified reaction zones of an array of reaction zones such that the chemical compositions react with the solid support(s) contained therein to modify the solid support or a composition attached to the solid support. Different reaction zones receive different chemical compositions in the diversity introducing reaction steps of the methods of the present invention so that different chemical compositions are produced on the solid supports located in different reaction zones. Libraries of compounds typically can be synthesized using between 2 and 100 reaction steps. In preferred applications, the number of diversity introducing reaction steps will be about the same as the dimensionality of the library, e.g., preferably there are between 2 and 10 diversity introducing reaction steps. More preferably there are between about 2 and about 8 diversity introducing reaction steps. In particularly preferred aspects there are about 3, 4, 5, 6 or 7 diversity introducing reaction steps. To generate a combinatorial library of such compounds, the synthons are introduced in “sets”, where the number of sets is about equal to the number of diversity introducing reaction steps required to make a compound of the library. Libraries of compounds typically can be synthesized using between about 2 and about 5000 synthons and preferably using between about 2 and about 1000 synthons. In particularly preferred embodiments, libraries of compounds are prepared using about 2 to about 100 synthons. Therefore, to synthesize a library of compounds where each compound is synthesized using 3 diversity introducing reaction steps, the methods use 3 sets of synthons. The synthons for a diversity introducing reaction step may be selected to react in a polymeric fashion to form a linear molecule having a structure specified by the identity of the building block at each position. Alternatively, the synthons for a diversity introducing reaction step may be selected to react in an interlocking manner, giving rise to non-linear three-dimensional structures.

### BRIEF DESCRIPTION OF THE DRAWING

For a fuller understanding of the nature and desired objects of the present invention, reference is made to the following detailed description taken in conjunction with the accompanying drawing figures wherein like reference character denote corresponding parts throughout the several views and wherein:

FIG. 1 is an exploded schematic diagram of a three dimensional reaction block that comprises 9 reaction columns arranged in a rectangular array and each reaction column contains 4 reaction zones arranged in a vertical stack each reaction zone containing a solid support;

FIG. 2 is an exploded schematic diagram of a three-dimensional reaction block that comprises four reaction planes with 16 reaction zones per reaction plane;

FIG. 3 is a schematic diagram of a 3-dimensional array of 27 three-dimensional reaction blocks giving a six-dimensional array of reaction zones;

FIG. 4 is a schematic diagram of a three-dimensional reaction block shown in the array of reaction blocks shown in FIG. 3 where each reaction block corresponds to the reaction blocks of FIG. 2;

FIG. 5 is a schematic diagram of a three-dimensional reaction block shown in the array of reaction blocks shown in FIG. 3 where each reaction block corresponds to the reaction blocks of FIG. 1;

FIG. 6 is a composite reaction block reaction plates with common (x, y) coordinates and disparate (z, z′) coordinates are stacked for a given (x′, y′) coordinate; and

FIG. 7 is a six dimensional array of reaction zones that comprises a two-dimensional array of composite reaction blocks as shown in FIG. 6.

### DESCRIPTION OF THE PREFERRED EMBODIMENT

The invention provides systems and methods for synthesizing chemical compounds by sequential addition of chemical building blocks onto solid supports in a parallel manner to produce a library of chemical compounds. The solid supports are in “reaction zones”, with a single compound synthesized in each reaction zone. The maximum number of different compounds that can be synthesized is thus equal to the number of reaction zones where a compound of the library can optionally include one or more regioisomers, diastereomers, enantiomers, conformers, geometric isomers, tautomers and other types of isomers. The reaction zones are typically arranged in a 3-dimensional reaction block, and are preferably maintained at fixed positions relative to one another during synthesis. Three-dimensional combinatorial libraries of compounds can be prepared in a system comprising a single reaction block. More complex combinatorial libraries of compounds are suitably prepared by using a system of the invention that comprises a plurality of reaction blocks that are typically arranged in one or more dimensions. Preferably the reaction blocks are arranged in one, two or three-dimensions such that the reaction zones are arranged in four, five or six-dimensions. Particularly preferred arrays of reaction blocks include arrays wherein the reaction blocks are arranged in one or two dimensions such that the reaction zones are arranged in four, five or more dimensions. An important feature of the invention is that the synthetic history of a solid support in a particular reaction zone is determined simply from the relative location of that reaction zone in the multi-dimensional array, e.g. the position of a reaction zone in the library of chemical compounds. Moreover the synthetic history, e.g., the combination of synthons contacted with a reaction zone solid support in the diversity introducing reaction steps, is determined by the position of the reaction zone in a reaction block and the position of the reaction block in a reaction assembly and the synthetic history is defined by three coordinates that locate the reaction zone in a reaction block and zero, one, two, three or more coordinates to locate the reaction block in the reaction assembly. In this way, the need to encode the individual supports is eliminated.

A reaction zone is defined as a space comprising at least one solid support that is uniquely defined by three or more coordinates. Typically a reaction block comprises a three dimensional array of reaction zones. Preferred reaction blocks comprise sufficient reaction zones so that at least one reaction zone of each possible reaction product resulting from all combinations of three diversity introducing reaction steps is included in the reaction block. In a typical embodiment a reaction block will comprise all possible combinations of the diversity introducing reaction steps that are varied along the x, y and z coordinates. However other combinations of diversity introducing reaction steps can also be contained in a reaction block such as (x, y, z′), (x′, y′, z), (x′, y′, z′) and the like. Reaction zones are in fluid contact with adjacent reaction zones that have common (x, y) coordinates but different z coordinate values, e.g., reaction zones that are arranged in a vertical stack. Further reaction zones with a common z coordinate but different (x, y) coordinates are fluidly separate such that reagents introduced into a vertical stack of reaction zones with a common (x, y) coordinate do not contaminate reaction zones with a different (x, y) coordinate. Different reagents and/or solutions can be introduced into the vertical stacks of reaction zones so that the reagents and/or solutions do not cross contaminate reaction zones that should receive different reagents or solutions.

A reaction assembly is defined herein as a plurality of reaction zones necessary to contain all the compounds of a library of compounds prepared by the methods of the present invention. A reaction assembly comprises at least as many reaction zones as there are compounds in the library of compounds to be prepared therein and the reaction zones of the reaction assembly are arranged in at least as many dimensions as the dimensionality of the library of compounds.

A reaction column is defined as a vertical stack of reaction zones that are in fluid contact and have a common (x, y) coordinate but different z coordinates. Addition of a reagent or solution to a reaction column results in the reagent or solution being introduced into all the reaction zones contained in the reaction column.

A synthon is defined as a chemical compound that reacts with one or more functional groups or chemical entities present on a solid support to form a new synthetic intermediate bound to the solid support. A synthon can be used in combination with other synthons or reagents such that different reaction zones of the reaction assembly receive different synthon compositions in a synthetic step that introduced synthetic diversity to the reaction zones of the assembly. See for example, D. Maclean, J. J. Baldwin, V. T. Ivanov, Y. Kato, A. Shaw, P. Schneider, E. M. Gordon; Journal of Combinatorial Chemistry, (2000), v. 2, no. 6, p. 562, for a definition for terms commonly used for combinatorial chemistry.

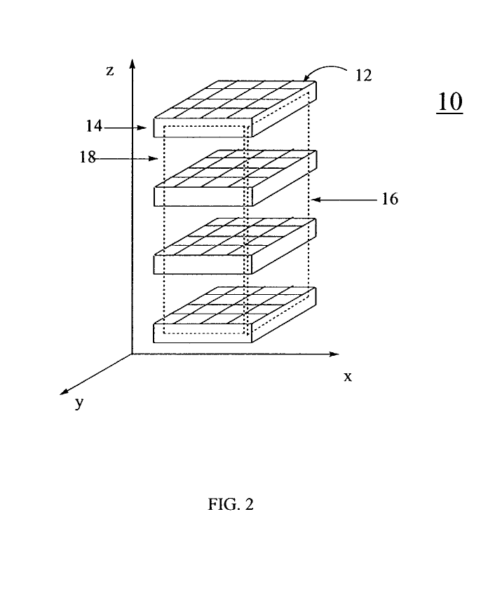

Referring now to the various figures of the drawing wherein like reference characters refer to like parts, there is shown in FIG. 2 a schematic diagram of a reaction zone assembly 10. Reaction zone assembly 10 includes a three-dimensional (4.times.4.times.4) array of reaction zones 12. However, it will be appreciated that such a number of reaction zones are set forth merely for purposes of illustration, and any number of reaction zones which are arranged in a three dimensional array may be used according to the principles of the present invention. For convenience of discussion, reaction zone assembly 10 may be provided with an x, y, z coordinate system, and may be described in terms of two-dimensional arrays or “reaction planes” of reaction zones. Using such a coordinate system, reaction zone assembly 10 may be divided into four horizontal (x, y) reaction planes 14, each of which includes a two dimensional array of 16 reaction zones. In a similar manner, reaction zone assembly 10 may be divided into four vertical (y, z) reaction planes 16 and four vertical (x, z) reaction planes 18. Each of reaction planes 16 and 18 also includes a two dimensional array of 16 reaction zones. Further, it will be appreciated that reactions zones 12 in planes 16 and 18 are arranged in 4 columns of 4 reaction zones per column. Each column contains reaction zones having common (x, y) but different z coordinates.

The use of a three dimensional array of reaction zones allows a different combination of chemical reagents or building blocks to be contacted or reacted with the supports in each (x, z) and (y, z) reaction plane. If the solid supports located in the reaction zones in each (x, y) plane are pre-derivatized with a different first diversity introducing reaction step, the resulting library will have a number of combinatorial compounds which is equal in number to the number of reaction zones. For example, since reaction zone assembly 10 of FIG. 2 includes a 4×4×4 array of reaction zones, the maximum number of chemical compounds that may be produced is 4³ or 64. Similarly using ten (10) 96 well filter bottom plates, which are stacked vertically, e.g., an 8×12×10 array of reaction zones, a library with 960 chemical compounds can be prepared.

One exemplary method for producing such a combinatorial collection of compounds using reaction zone assembly 10 will next be described. For convenience of discussion, the method described is one where the maximum number of combinatorial compounds is produced (i.e., a number equal to the number of reaction zones). However, it will be appreciated that fewer compounds may be produced by simply duplicating one or more of the chemicals or building blocks that are introduced into the reaction zone planes.

In the method, each of the reaction planes is provided during synthesis with a different combination of at least three sets of synthons, such as 3, 4, 5 or 6 sets of synthons, to produce 4³ or 4ⁿ chemical combinations where n is the number of diversity introducing reaction steps. Each reaction zone contains a solid support, which is preferably pre-derivatized by one of four different first diversity introducing reaction step chemical compositions. For convenience, a plurality of solid supports (in this example at least 16) can be contacted with each chemical composition of the first diversity introducing reaction step in four separate reactions using standard combinatorial chemistry techniques prior to distribution of the solid supports to the reaction zones in each reaction plane. The four sets of different solid supports with different synthetic histories from the first diversity introducing reaction step are typically distributed such that all reaction zones in a (x, y) reaction plane 14 contain supports pre-derivatized with the same first chemical composition. Similarly, the other (x, y) reaction planes are uniformly provided with a different first chemical composition. In this way, reaction zone assembly 10 will initially be provided with 64 supports having four different chemical building blocks derivatized thereto.

A second diversity introducing reaction step is then carried out by introducing into the reaction zones of each of the (y, z) reaction planes a different second diversity introducing reaction step chemical composition, such that supports in all zones having a common x coordinate value are contacted with the same second chemical composition. The second diversity introducing reaction step typically occurs under conditions that result in the formation of a compound synthesized from the reaction of the first and second diversity introducing reaction steps so that there are 16 sets of 4 supports with a common reaction history. In the final step, a third diversity introducing reaction step chemical composition is carried out by introducing into the reaction zones of each of the (y, z) reaction planes, such that supports in all zones having a common y coordinate value are contacted with the same third chemical composition. As above, the third diversity introducing reaction step typically occurs under conditions that result in the formation of a compound synthesized from the reation of the first, second and third diversity introducing reaction steps. If different chemical compositions are used in the different reaction planes for the first, second and third diversity introducing reaction steps, the library comprises 64 solid supports wherein each support has a different synthetic history in each of the reaction zones.

Of course, some of the chemicals may be duplicated so that the total number of chemical combinations will be less than the number of reaction zones. Further, it will be appreciated that each support may be contacted by more than or less than three diversity introducing reaction steps to produce other kinds of combinatorial libraries including partial libraries of combinatorial libraries of compounds that are prepared with four or more building blocks. For example, all of the solid supports may be derivatized with a common building block in the first step, then split into n reaction vessels and reacted with n building blocks in a second step before being distributed into the reaction zones of an (x, y) reaction plane.

Additional reagents and chemicals can be introduced into some or all of the reaction zones of the reaction block between the steps of contacting the compounds bound to the solid support with the diversity introducing reaction steps. In particular, reagents or chemicals used in the protection and/or deprotection of functional groups can be contacted with solid support of the array to protect or deprotect one or more functional groups. Techniques for the protection and deprotection of functional groups are well established in the art, see for example, Green, T. W. and Wuts, P. G. M., Protective Groups in Organic Synthesis, John Wiley & Sons, New York, 1991. Additionally, reagents for functional group transformations can be introduced in combination or separately from the chemical compositions of the diversity introducing reaction steps, for example reagents can be introduced for oxidation, reduction, hydrolysis and other types of functional group transformations. Further, chemicals and reagents can be contacted with some or all of the solid supports of the library either in concert or sequentially, to cleave compounds of the library of compounds from the solid support. Cleavage reactions can suitably occur as a separate reaction step or concomitantly with the last diversity introducing reaction step.

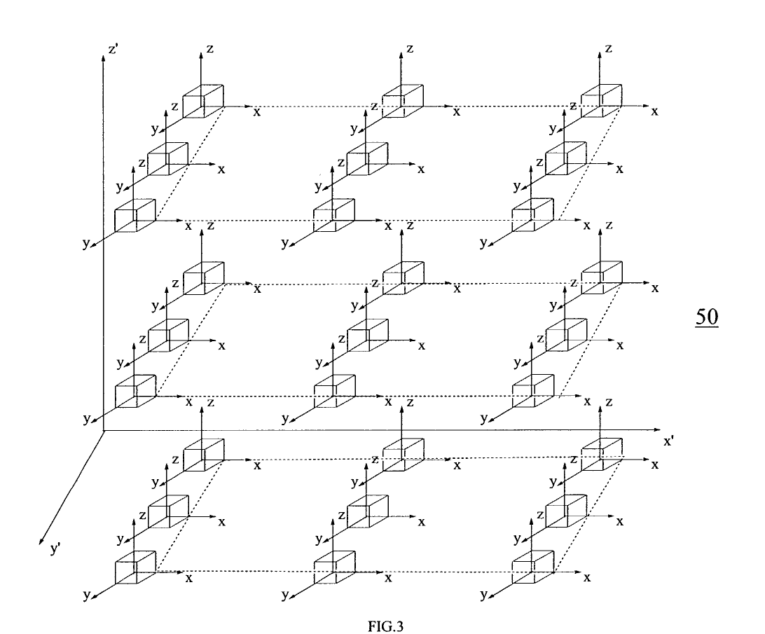

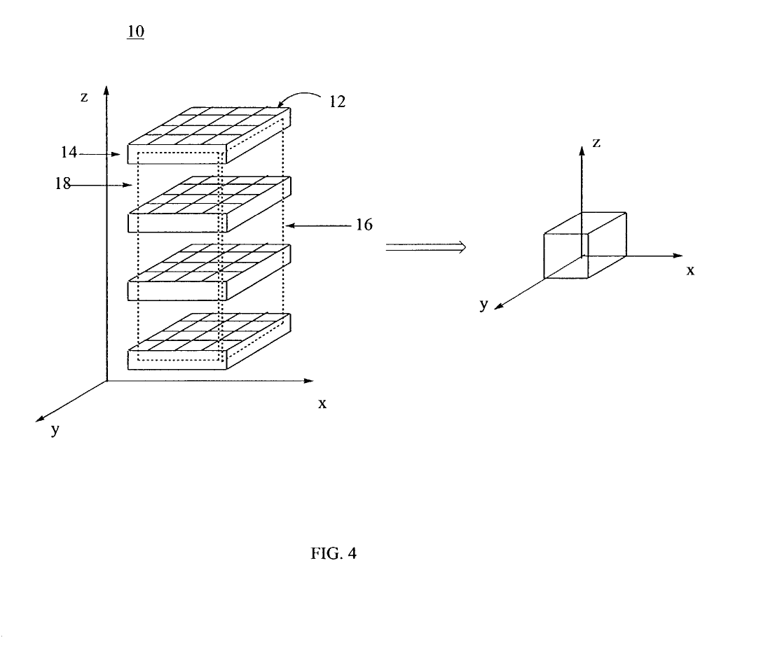

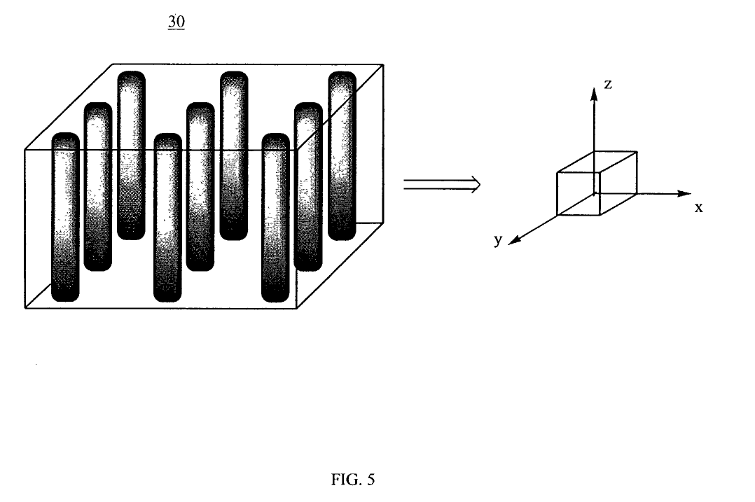

There is shown in FIG. 3 a schematic diagram of a reaction zone assembly 50 that comprises a 3×3×3 array of the above described reaction block 10, e.g., a reaction block with 64 reaction zones arrayed in three dimensions. There is shown in FIG. 4 a schematic drawing of a reaction block 10 as it relates to the reaction block of the reaction assembly 50. Similarly, there is shown in FIG. 5 a schematic drawing of a reaction block 30 as it relates to the reaction block of the reaction assembly 50. It will be appreciated that such a number of reaction blocks 10 and the number of reaction zones 12 in each reaction block are set forth merely for purposes of illustration, and any number of reaction blocks comprising any number of reaction zones per reaction block may be used according to the principles of the present invention. Moreover, any zero-, one-, two- or three-dimensional array of reaction blocks corresponding to a three-, four-, five- or six-dimensional library of reaction zones may be used according to the principles of the present invention. For convenience of discussion, the reaction assembly 50 may be provided with an x, y, z coordinate system to identify a specific reaction zone 12 in a reaction block 10 or reaction block 30 and may be further provided with an x′, y′, z′ coordinate system to identify a specific reaction block 10 or reaction block 30 in the reaction assembly 50. The reaction assembly 50 may be described in terms of two-dimensional arrays or “reaction block planes” of reaction blocks 10 or reaction blocks 30. Using such a coordinate system, reaction assembly 50 may be divided into three horizontal (x′, y′) reaction block planes, each of which includes a two-dimensional array of 9 reaction blocks. In a similar manner, reaction assembly 50 may be divided into three vertical (y′, z′) reaction planes and three vertical (x′, z′) reaction planes. Each of the reaction block planes also includes a two-dimensional array of 9 reaction blocks. Further it will be appreciated that reaction blocks at the intersection of a (x′, z′) reaction block plane and a (y′, z′) reaction block plane form a column of three reaction blocks where each reaction block has common (x′, y′) coordinates but different (z′) coordinates.

One exemplary method for producing such a six-dimensional combinatorial collection of compounds using reaction zone assembly 10 will next be discussed for a 3×3×3 array of reaction blocks where each reaction block is a 4×4×4 array of reaction zones such that the method will produce a combinatorial library of 4×4×4×3×3×3 or 4³×3³ or 1728 chemical combinations. For convenience of discussion, the method described is one where the maximum number of combinatorial compounds is produced, i.e., a number equal to the number of reaction zones. However, it will be appreciated that fewer compounds may be produced simply by duplicating one or more of the chemical compositions or diversity introducing reaction steps that are contacted with the solid supports of the reaction zones or reaction blocks of the array.

In the method, each of the reaction blocks is provided during synthesis with all possible combinations of the first, second and third diversity introducing reaction steps, e.g., 64 or 4³ solid supports with different synthetic histories, such that each reaction block has a common synthetic history in each reaction zone with common (x, y, z) coordinates. 27 reaction blocks 10 are prepared according to the above-described method for preparing a three dimensional library of compounds in a single reaction block 10. Different fourth diversity introducing reaction step chemical compositions are then introduced into the reaction zones 12 of all the reaction blocks 10 of each (x′, y′) reaction block planes, such that the solid supports in all reaction zones 12 having a common z′ coordinate value are contacted with the same fourth chemical composition. The fourth diversity introducing reaction step typically occurs under conditions conducive to the formation of a compound synthesized from the first, second, third and fourth diversity introducing reaction steps.

A different fifth diversity introducing reaction step chemical composition is then introduced into the reaction blocks of each of the (y′, z′) reaction block planes, such that supports in all zones having a common x′ coordinate value are contacted with the same fifth chemical composition. The fifth diversity introducing reaction step typically occurs under conditions conducive to the formation of a compound synthesized from the first, second, third, fourth and fifth diversity introducing reaction steps.

In a final step, a different sixth diversity introducing reaction step chemical composition is introduced into the reaction zones of each of the (x′, z′) reaction block planes, such that supports in all zones having a common y′ coordinate value are contacted with the same sixth chemical composition. As above, the sixth diversity introducing reaction step typically occurs under conditions conducive to the formation of a compound synthesized from the first, second, third, fourth, fifth and sixth diversity introducing reaction steps.

In certain embodiments, an additional diversity introducing reaction step can be introduced without expanding the dimensionality of the reaction assembly. For example, the last diversity introducing reaction step can suitably be effected under conditions suitable to cleave the final product from the solid support and each reaction zone of the reaction assembly comprises at least as many solid supports as different chemical compositions of the last diversity introducing reaction step. Preferably, methods in which the final combinatorial chemical reaction and cleavage reaction are accomplished concomitantly, the reaction blocks of the reaction assembly are disassembled prior to contacting the last diversity introducing reaction step chemical composition with the solid supports which have a synthetic history resulting from the previous diversity introducing reaction steps so that the solid supports contained in individual (x, y, z, x′, y′, z′) reaction zones 12 are not in fluid contact with adjacent reaction zones in the z direction. At least one solid support from each reaction zone is separately contacted with a different chemical composition of the last diversity introducing reaction step. In this way, a six dimensional library of chemical compounds can be prepared in a five dimensional reaction assembly, a seven dimensional library of chemical compounds can be prepared in a six dimensional reaction assembly and so forth.

Additional reagents and chemicals can be introduced into some or all of the reaction zones 12 of a reaction block 10 of the array of reaction blocks, e.g., the reaction assembly 50, or to all of the reaction zones of one or more reaction blocks 10 of the reaction assembly 50 between the steps of contacting the solid supports with the chemical compositions of the diversity introducing reaction steps. In particular, reagents or chemicals used in the protection or deprotection of functional groups and in the transformation of a functional group into another type of functional group can be contacted with the solid supports in the appropriate reaction zones of the reaction block 10 to protect, deprotect or transform into another functional group one or more functional groups. Further, chemicals and reagents can be contacted with some or all of the reaction zones of a reaction plane, to cleave the compound of the combinatorial library from the solid support. The cleavage reaction can suitably occur as a separate reaction or concomitantly with the final diversity introducing reaction step.

Each reaction block may be identified in the three-dimensional array of reaction blocks by coordinates x′, y′, z′, which correspond to Cartesian coordinates. However, it will be appreciated that such a number of reaction zones and reaction blocks are set forth merely for purposes of illustration, and any number of reaction zones 12, which are arranged in a six-dimensional array, may be used according to the principles of the present invention. It will further be appreciated that a reaction assembly 50 in which the maximum coordinate value for one or two of the coordinates x′, y′ and/or z′ is one results in a two or one-dimensional array of reaction blocks 60. When, for example, z′=1 for all reaction blocks 60 of the reaction assembly 50, then a five-dimensional reaction assembly 50 is generated having the coordinates (x, y, z, x′, y′, 1) or (x, y, z, x′, y′). Similarly, when, for example, y′=1 and z′=1 for all reaction blocks 60 of the reaction assembly 50, then a four-dimensional reaction assembly 50 is generated having the coordinates (x, y, z, x′, 1, 1) or (x, y, z, x′). Similarly, a reaction assembly 50 that includes only one reaction block 60, e.g., x′=1, y′=1 and z′=1 results in a three-dimensional reaction zone assembly 10.

In preferred embodiments of the present invention, a three dimensional library of compounds may be prepared in a reaction block comprising a two dimensional array of reaction columns, e.g. a square or rectangular array, having x and y coordinates of reaction columns where each reaction column has two or more reaction zones stacked in the z coordinate direction. More specifically, each reaction zone has at least one solid support such that the solid support of a reaction zone can not mix or exchange position with the supports of adjacent reaction zones in the z coordinate. Preferably, each reaction zone has a single solid support and the solid support is chosen such that two solid supports cannot exchange places in the reaction column. More specifically, the z coordinate for a solid support of a reaction zone is invariant during the preparation and storage of the library of compounds in the reaction block.

Suitable solid supports include lanterns™, CD plugs, Irori kans™, synthesis resin beads and the like. Suitable reaction blocks include an array of glass tubes, filter syringes, Robbins block wells, Bohdan mini-blocks™, Charybdis Calypso™ blocks or Radley's Combiclamp™ or like columns arranged in a square or rectangular array in a suitable support base or rack.

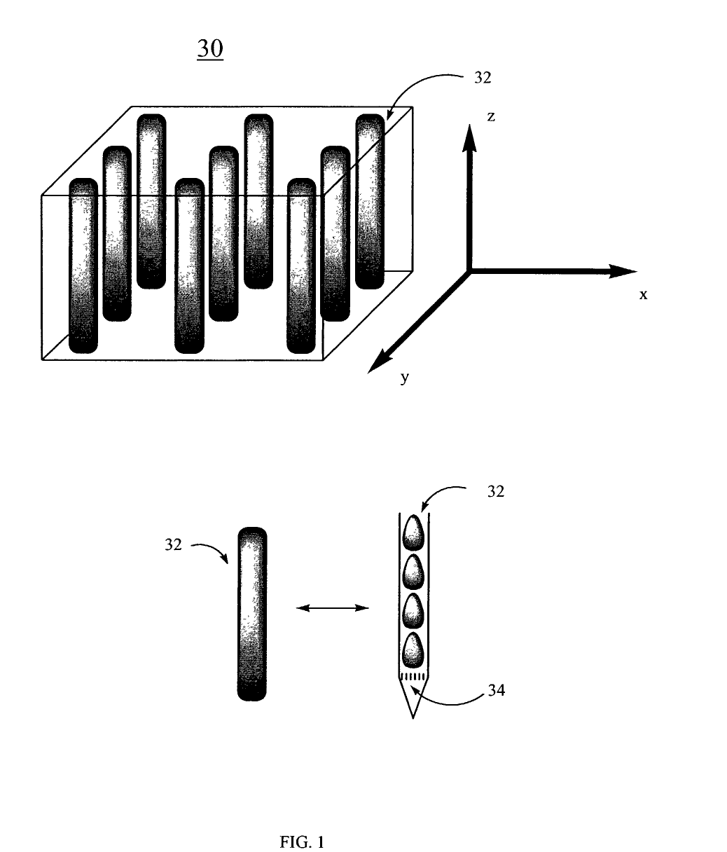

Now referring to FIG. 1, a reaction block 30 that comprises a two-dimensional array of reaction columns 32 can be prepared by the following method. The method including the steps of: (i) arranging a plurality of empty reaction columns 32 in a two-dimensional, e.g. square or rectangular, array having x and y coordinates; (ii) pre-derivatizing a sufficient number of solid supports with each first diversity introducing reaction step chemical compositions by contacting separate batches of solid supports with a different first chemical composition under conditions conducive to formation of solid supports with a synthetic history corresponding to the first diversity introducing reaction step; (iii) introducing solid supports into each reaction column of the two-dimensional array of reaction columns to form a reaction plane of reaction zones 12 in the (x, y) plane where each solid support has a common synthetic history, e.g., the first diversity introducing reaction step chemical composition corresponds to z=1; (iv) introducing a solid support into each reaction column 32 of the two-dimensional array of reaction columns 32 to form a reaction plane of reaction zones 12 in the (x, y) plane where each solid support has a common synthetic history that is different from the synthetic history of the solid supports of step (iii), e.g., the first diversity introducing reaction steps corresponding to z=2; (v) repeating step (iv) until the reaction block has an (x, y) reaction plane of reaction zones 12 of solid supports with a synthetic history for all possible first diversity introducing reaction step chemical compositions.

A second diversity introducing reaction step chemical composition is then introduced into the reaction zones 12 of each of the (y, z) reaction planes, such that supports in all zones having a common x coordinate value are contacted with the same second chemical composition. The second diversity introducing reaction step typically occurs under conditions conducive to the formation of a compound synthesized from the first and second diversity introducing reaction steps.

In the final combinatorial step, a third diversity introducing reaction step chemical composition is introduced into the reaction zones 12 of each of the (y, z) reaction planes, such that supports in all zones having a common y coordinate value (i.e., (y, z) planes) are contacted with the same third chemical composition. As above, the third diversity introducing reaction step typically occurs under conditions conducive to the formation of a compound synthesized from the first, second and third diversity introducing reaction steps. If different chemical compositions are used in the different reaction planes for each diversity introducing reaction step as described above, the method generally results in the formation of a solid support with a different synthetic history in each reaction zone.

The compounds of the synthesized chemical library can be isolated from the reaction block 30 by the following process. The top reaction plane or layer of reaction zones 12 is removed from the reaction block 30 and placed in a separate two-dimensional array, e.g., a square or rectangular array. Preferably, each two-dimensional array is appropriately marked such that the location of a reaction plane in the reaction block 30 from which the solid supports are transferred is clearly denoted. The solid support from each reaction zone 12 is transferred to the two-dimensional array, such that the x, y coordinate of the reaction zones from the reaction block 30 corresponded with the x, y coordinate of the receiving array of solid supports, e.g. the solid support from reaction tube (x, y)=(1, 1) of the reaction block 30 was placed in position (x, y)=(1, 1) of the receiving rack, and so on such that the solid support from reaction tube (x, y)=(X, Y) of the reaction block 30 is placed in well (x, y)=(X, Y) of the receiving rack. This process is repeated, such that the solid supports from each reaction plane are placed into the corresponding positions in receiving racks that are suitably marked to denote the (x, y) coordinates of each solid support and the reaction plane of origin, e.g. the z coordinate.

A plurality of reaction blocks 30, each of which comprise a two-dimensional array of reaction columns 32, can be used to form a four, five or six dimensional combinatorial library of chemical compounds in a reaction assembly 50 as depicted in FIG. 4. A plurality of three-dimensional reaction blocks 30 can be prepared wherein reaction zones 12 with a common (x, y, z) coordinate contain a common solid support bound chemical composition. These reaction blocks 30 are then arranged in a one, two, or three-dimensional array of reaction blocks having (x′), (x′, y′), or (x′, y′, z′) coordinates to form a four, five or six dimensional array of reaction zones. Alternatively, a single reaction block can be employed provided that the reaction block has sufficient wells to house all XYZX′Y′Z reaction zones of the library of chemical compounds.

For a six-dimensional library of compounds, the three-dimensional array of reaction blocks 50 is further functionalized by contacting the reaction zones 12 of each reaction block 30 with a different combination of fourth, fifth and sixth diversity introducing reaction step chemical compositions.

A different fourth diversity introducing reaction step chemical composition is introduced into the reaction blocks 30 of each of the (x′, y′) reaction block planes, such that supports in all zones having a common z′ coordinate value are contacted with the same fourth chemical composition. The fourth diversity introducing reaction step typically occurs under conditions conducive to the formation of a compound synthesized from the first, second, third and fourth diversity introducing reaction steps.

A fifth diversity introducing reaction step chemical composition is then introduced into the reaction blocks 30 of each of the (y′, z′) reaction block planes, such that supports in all zones having a common x′ coordinate value are contacted with the same fifth chemical composition. The fifth diversity introducing reaction step typically occurs under conditions conducive to the formation of a compound synthesized from the first, second, third, fourth and fifth diversity introducing reaction steps.

In a final step, a different sixth diversity introducing reaction step chemical composition is introduced into the reaction blocks 30 of each of the (x′, z′) reaction block planes, such that supports in all zones having a common y′ coordinate value are contacted with the same sixth chemical composition. As above, the sixth diversity introducing reaction step typically occurs under conditions conducive to the formation of a compound synthesized from the first, second, third, fourth, fifth and sixth diversity introducing reaction steps.

In other preferred embodiments, a 4, 5 or 6 dimensional library where each reaction zone of the library is uniquely identified by 4, 5 or 6 coordinates can be prepared in one or more composite reaction blocks 70. Each composite reaction block 70 has at least one composite axis that is dependent upon two coordinates of the library, e.g., the coordinate values for x and x′, y and y′, and/or z and z′ are combined to form a composite coordinate. For example, values of composite coordinates a, b and c are determined from the values of x and x′, e.g., a=X(x′−1)+x, y and y′, e.g., b=Y(y′−1)+y, or z and z′, e.g., c=Z(z′−1)+z, where X, Y, and Z are the number of different compositions for each diversity introducing reaction step, e.g., a reaction or process that introduces diversity into the library of compounds, which is varied along the x, y and z axis of the library. A composite reaction block 70 preferably comprises all (x, y, z) reaction zones that will receive different compositions for a diversity introducing reaction step that introduces diversity into the library of compounds according to the one or more of the x′, y′, z′ coordinates of the multidimensional axis of the library.

Preferably the library can be prepared in one composite reaction block that comprises all the reaction zones of the assembly. Further the composite reaction block has the reaction zones arranged in a rectangular prismatic array with axes defining three composite coordinates for a six dimensional library, e.g., a, b and c wherein:

a=X(x′−1)+x

b=Y(y′−1)+y

c=Z(z′−1)+z′

Using the a, b and c composite coordinates defined above results in a composite reaction block wherein there are regions of the block with reaction zones with reaction histories that have different x, y and z coordinates and common x′, y′, and z′ coordinates. Clearly, the organization of the reaction block is arbitrary and any other arrangement of reaction zones into regions with different common coordinates are also suitable for use with the present invention.

Two or more reaction blocks of a four, five or six-dimensional array can be combined to form larger reaction blocks to facilitate the addition of appropriate reagents and building blocks to each reaction zone of the library array. Alternatively, the dimensions of a single reaction block can be extended in the x, y and z dimensions depending on the number of variables, e.g., reactions that introduce diversity into the library, within the reaction steps that introduce diversity.

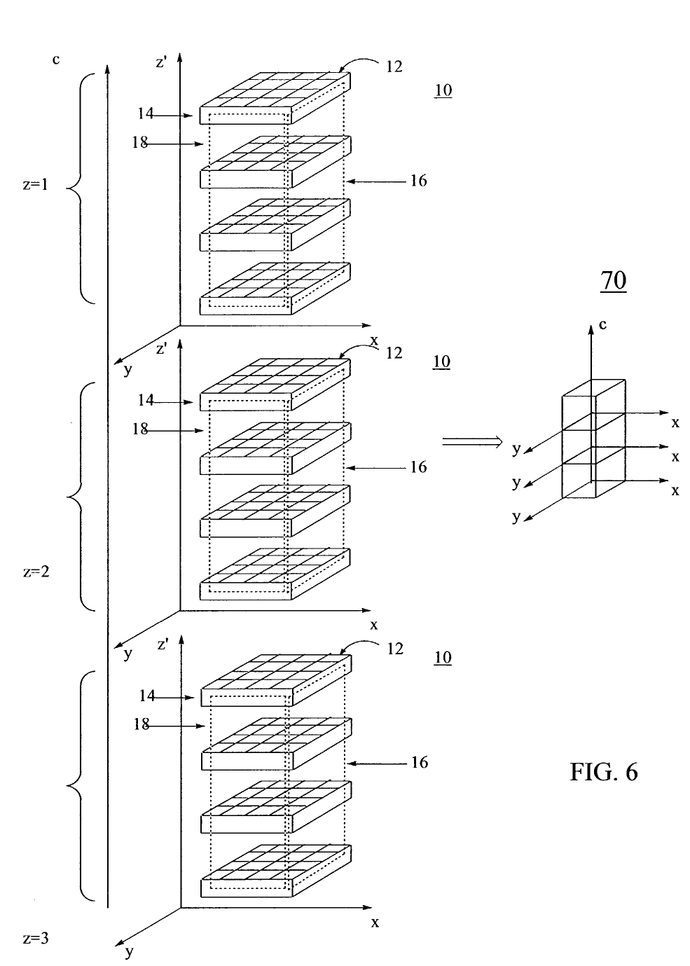

There is shown in FIG. 6 a schematic diagram of a composite reaction block 70 comprising three reaction blocks 10. The vertical arrangement of the reaction blocks 10 in the composite reaction block 70 has been arbitrarily selected for convenience of illustration. Other composite reaction blocks 70 are also contemplated to be within the scope of the present invention. In preferred embodiments, composite reaction blocks 70 are prepared by horizontally combining at least two reaction blocks 10. Preferably a sufficient number of reaction blocks 10 are combined horizontally to form a composite block with all possible values of a, b, c or a combination thereof within a single composite reaction block 70.

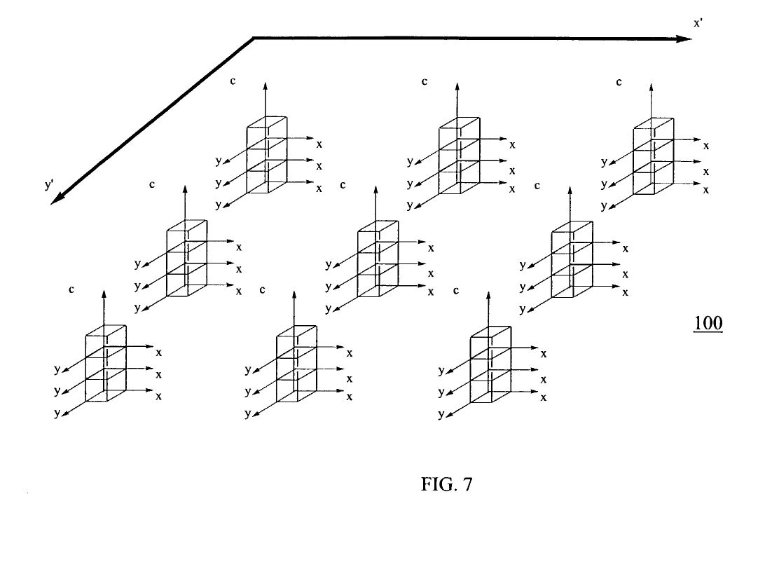

For example, there is shown in FIG. 7 a six-dimensional reaction assembly 100 comprising a two dimensional array of reaction blocks 70 as depicted in FIG. 6. Each reaction block 70 comprises all reaction zones with a common (x′, y′) coordinates, e.g., (x, y, c, x′, y′), so that the composite reaction blocks 70 with different x′ and y′ coordinates are arranged in a two-dimensional array, e.g., a square or rectangle, and all possible (x, y, z, x′, y′, z′) combinations are arranged in reaction zones according to (x, y, c, x′, y′) reaction coordinates.

An illustrative method for preparing a six-dimensional library of compounds using the reaction assembly 100 depicted in FIG. 7 includes preparing a plurality of reaction blocks 70 with solid supports with all possible synthetic histories in reaction zones uniquely identified by their (x, y, c) coordinate values. Each composite reaction block 70 of the reaction assembly 100 then receives the appropriate combination of chemical compositions for the diversity introducing reaction steps that are varied along the x′ and y′ coordinate axes.

In a similar example for a five-dimensional library of compounds, reaction blocks with a common x′ coordinate can be combined in so that reaction blocks with the same x′ coordinate but different y′ coordinate are vertically stacked and are in fluid contact with other reaction zones which have a common x′ coordinate. The building block that is varied along the x′ axis is then contacted with the appropriate building block. The larger reaction blocks are then disassembled and the fifth building block introduced in to the reaction blocks with the corresponding y′ coordinate value.

There is shown in FIG. 7 a reaction assembly 100 with a six dimensional array of reaction zones arranged in composite reaction blocks 70 where each reaction zone is uniquely identified by its (x, y, c, x′, y′) coordinates. The c composite coordinate is dependent upon two coordinates of the six dimensional library of chemical compounds, e.g., c=Z(z′−1)+z where Z is the number of different chemical compositions of the diversity introducing reaction step that is varied along the z coordinate of the library.

There is shown in FIG. 7, a schematic diagram of a reaction zone assembly 100 that comprises an array of composite reaction blocks 70 arranged in a two-dimensional array. Further, as shown in FIG. 6, each composite reaction block 70 of the assembly 100 comprises ZZ′ or C reaction planes where Z is the number of different chemical compositions of the diversity introducing reaction step that is varied along the z coordinate of the library and Z′ is the number of different chemical compositions of the diversity introducing reaction step that is varied along the z′ coordinate of the library. Typically, the last diversity introducing reaction step is the sixth diversity introducing reaction step for a six-dimensional library of compounds, the fifth diversity introducing reaction step for a five-dimensional library of compounds or the fourth diversity introducing reaction step for a four-dimensional library of compounds. Further, a four-dimensional library of compounds generally can be prepared with a single (x, y, c) composite reaction block 70; a five-dimensional library of compounds can be prepared with a linear array of (x, y, c) composite reaction blocks 70; and a six-dimensional library of compounds can be prepared with a two-dimensional array, e.g., a square or rectangular array, of (x, y, c) composite reaction blocks 70. Other composite reaction blocks, such as a five-dimensional (a, b, z) composite reaction block or arrays of other composite reaction blocks will be suitable for the preparation of libraries of compounds and are contemplated in the present invention.

For the purposes of illustration, a two-dimensional array of reaction blocks 70 is depicted in FIG. 7, but it is readily apparent that one or both of x′ and/or y′ can be limited to a maximum value of one such that the two-dimensional array of reaction blocks can be limited to a one or zero-dimension array of reaction blocks such that the assembly 100 is appropriate for the preparation of a five or four-dimensional combinatorial library of compounds. Further, it will be appreciated that a three dimensional array of reaction blocks 70 results in an assembly 100 that is appropriate for the preparation of a seven-dimensional combinatorial library of compounds.

A reaction block 70 can be organized such that the reaction planes that comprise a common first building block are grouped together. The reaction block has a composite variable c equal to Z′(z−1)+z′ wherein Z′ is the number of different last building blocks, z and z′ are the coordinates defining the first and last building block of the combinatorial library of compounds. However other sets of reaction coordinates, e.g., coordinates of the library, and other diversity introducing reaction steps can be combined into composite coordinates and such reaction assemblies and libraries are also contemplated in the present invention.

Alternatively, a reaction block 70 can be organized such that the reaction planes that comprise a common last building block are grouped together. The reaction block has a composite variable c equal to Z(z′−1)+z wherein Z is the number of different last diversity introducing reaction step chemical compositions, z and z′ are the coordinates defining the first and last diversity introducing reaction steps of the library of compounds.

In a preferred embodiment of the present invention, a six-dimensional combinatorial library of compounds is prepared from a two-dimensional array of reaction blocks. Each (x, y, c) composite reaction block 70 comprises ZZ′ reaction planes, e.g. Z′ reaction planes where each of the Z′ reaction planes has a common first diversity introducing reaction step chemical composition bound to the solid support. Preferably, the reaction block comprises Z domains where each domain includes Z′ adjacent reaction planes that comprise solid supports with a common first diversity introducing reaction step synthetic history. The variable c is a composite coordinate, e.g., c=Z′(z−1)+z′, dependant on both z and z′. A plurality of reaction blocks 70 are prepared such that corresponding reaction zones with common (x, y, c) coordinates in each composite reaction block of the reaction assembly 100 have a common first building block composition bound to the solid support. The reaction blocks are arranged in a two-dimensional array of reaction blocks having x′ and y′ coordinates.

One exemplary method for producing such a six-dimensional combinatorial collection of compounds using reaction zone assembly 100 will next be discussed for a 3×3 array of composite reaction blocks 70 where each reaction block comprises 12 reaction planes, e.g., Z=4, Z′=3 and ZZ′=12, and each reaction plane is a 4×4 array of reaction zones. The method will produce a combinatorial library of 1728, e.g., 4×4×4×3×3×3 or 4³×3³, chemical combinations. For convenience of discussion, the method described is one where the maximum number of combinatorial compounds is produced, i.e., a number equal to the number of reaction zones. However, it will be appreciated that fewer compounds may be produced simply by duplicating one or more of the chemical compositions or diversity introducing reaction steps that are introduced into the reaction zone planes or reaction block planes. For further convenience of discussion, the method described relates to reaction blocks in which all reaction planes with a common first building block component are adjacent to one another such that c=Z′(z−1)+z′. It will be appreciated that other reaction plane arrangements will also result in the formation of a library of compounds and such reaction plane arrangements are also acceptable for use in the methods of the present invention. In non-limiting examples, a library can be prepared in four-dimensional (x, y, c) composite blocks where c=Z(z′−1)+z or a library can be prepared in one or more five-dimensional (a, b, z) composite blocks where a=X(x′−1)+x and b=Y(y′−1)+y.

The method utilizing composite reaction blocks 70 includes the steps of: (i) providing a plurality of composite reaction blocks 70 that comprise solid supports with all possible synthetic histories resulting from the first, second and third diversity introducing reaction steps, such that each composite reaction block has three reaction zones with a common synthetic history. More specifically, the resulting composite reaction block 70 comprises 12 (x, y) reaction planes arranged in four sets of three equivalent reaction planes that have solid supports with a common synthetic history in equivalent (x, y) reaction zones.

The method of preparing the reaction blocks for the array comprises the steps of: (i) contacting in four separate reactions, batches of 432 solid supports (4×4×3×3×3) with one of four different first diversity introducing reaction step chemical compositions using standard combinatorial chemistry techniques; (ii) distributing the solid supports contacted with the first diversity introducing reaction step chemical composition corresponding to z=1 to each of the wells of all 9 reaction blocks to form three reaction planes, e.g. three solid supports are distributed to each reaction column of each reaction block to form three reaction planes; (iii) repeating step (ii) three times in succession for the solid supports derivatized with the first diversity introducing reaction step chemical compositions corresponding to z=2, z=3 and z=4. The solid supports which were contacted with the four different first diversity introducing reaction step chemical compositions are typically distributed such that all reaction zones in the top three (x, y) reaction planes 14 contain solid supports with a common synthetic history. Similarly, the next three (x, y) reaction planes disposed below the top three (x, y) reaction planes are uniformly provided with solid supports with a common synthetic history that is different from the synthetic history of the supports in the first three (x, y) reaction planes, and so on.

A second diversity introducing reaction step chemical composition is introduced into the reaction zones of each of the (y, z) reaction planes, such that the solid supports in reaction zones having a common x coordinate value are contacted with the same second chemical composition. The second diversity introducing reaction step typically occurs under conditions conducive to the formation of compound synthesized from the first and second diversity introducing reaction steps.

A third diversity introducing reaction step chemical composition is then introduced into the reaction zones of each of the (y, z) reaction planes, such that supports in all zones having a common y coordinate value (i.e., (y, z) planes) are contacted with the same third chemical composition. As above, the third diversity introducing reaction step typically occurs under conditions conducive to the formation of a compound synthesized from the first, second, and third diversity introducing reaction steps. If different diversity introducing reaction step chemical compositions are used in the different reaction planes as described above, the method results in the formation of solid supports with different synthetic histories in reaction zones with different (x, y, z) coordinate, e.g., there are three solid supports in each (x, y, z) composite reaction block with common (x, y, z) coordinates but different z′ coordinates.

A fourth diversity introducing reaction step chemical composition is then introduced into the reaction zones of each of the linear array of composite reaction blocks 70 with a common x′ coordinate such that reaction zones that have different x′ coordinates are contacted with different fourth chemical compositions. Moreover, reaction zones that have a common x′ coordinate are contacted with a common fourth chemical composition. The fourth diversity introducing reaction steps typically occurs under conditions conducive to the formation of a compound synthesized from the first, second, third and fourth diversity introducing reaction steps.

A fifth diversity introducing reaction step chemical composition is then introduced into the reaction zones of each linear array of composite reaction blocks with a common y′ coordinate such that reaction zones that have different y′ coordinates are contacted with different fifth chemical composition. Moreover, reaction zones that have a common y′ coordinate are contacted with a common fifth chemical composition. The fifth diversity introducing reaction step typically occurs under conditions conducive to the formation of a compound synthesized from the first, second, third, fourth and fifth diversity introducing reaction steps.

The reaction blocks are disassembled such that each set of reaction zones having the same (z, x′, y′, z′) are transferred to separate multiwell plates that are suitably marked to denote the (z, x′, y′, z′) coordinate values of the originating reaction plane such that the (x, y) coordinate of the reaction zones in the composite reaction block 70 generally corresponds with the (x, y) coordinate of the solid support in the separate multiwell plate. There are 108 separate multiwell plates corresponding to all combinations of (z, x′, y′, z′) and each multiwell plate has all possible (x, y) combinations for the (z, x′, y′, z′) coordinates of the reaction assembly 100 that were transferred to the multiwell plate.

The solid supports contained in each (z, x′, y′, z′) multiwell plate are contacted with a common sixth diversity introducing reaction step chemical composition such that all reaction zones with a common z′ coordinate value are contacted with the same sixth chemical composition. The sixth diversity introducing reaction steps typically occurs under conditions conducive to the formation of a compound synthesized from the first, second, third, fourth, fifth and sixth diversity introducing reaction steps. Cleavage of the compound of the library of compounds from the solid support may optionally occur concomitantly to the last diversity introducing reaction step or it may occur in a separate non-diversity introducing reaction step.

Some of the chemicals may be duplicated so that the total number of chemical combinations will be less than the number of reaction zones. Further, it will be appreciated that each support may receive more than or less than six diversity introducing reaction steps to produce other kinds of libraries of compounds or partial sets of more complex libraries of compounds.

When the method is performed with reaction blocks 70 that comprise a vertical stack of two-dimensional reaction plates, the disassembly of the reaction block does not have to include the transfer of the solid supports of the reaction block to separate multiwell plates. Instead, the reaction blocks are disassembled by simply unstacking the vertically stacked reaction plates. It will be appreciated that each reaction plate of each reaction block can be suitably marked to accurately identify each reaction zone of the reaction plate and identify the (z, x′, y′, z′) coordinates of the reaction plate. The solid support bound chemical compounds can then be contacted with the appropriate sixth diversity introducing reaction step chemical composition under conditions conducive for the formation of a compound synthesized by the first, second, third, fourth, fifth and sixth diversity introducing reaction steps and the cleavage of the compound from the solid support.

It will be further appreciated that a seven-dimensional combinatorial library of compounds can be prepared from a reaction assembly comprising a three-dimensional array of composite reaction blocks 70. Further, higher dimensionality libraries of compounds, e.g., libraries with seven, eight, or more dimensions, can suitably be prepared by the combination of one or more arrays and/or methods of the present invention and are considered within the scope of the present invention. These higher dimensional combinatorial libraries of compounds can be used particularly for the preparation of libraries of peptides and other oligiomeric and/or polymeric families of compounds.

The coordinates, x, y and z, define a specific reaction zone in a three-dimensional array of reaction zones, e.g., a reaction block. The coordinates, x and y, define a specified reaction zone in a two-dimensional reaction plane, and the reaction zones of a reaction plane are preferably organized in a regular Cartesian grid arrangement with rows and columns. There are preferably at least x rows and y columns so that all possible combinations of the second (x) and third (y) diversity introducing reaction step chemical compositions are contained in a single reaction plane. Two or more reaction planes, e.g. z planes are stacked vertically in a reaction block such that all reaction zones with a common z coordinate receive a common first diversity introducing reaction step chemical composition and are located in the same reaction plane. The reaction block comprises solid supports with synthetic histories arising from all possible combinations of the first, second and third diversity introducing reaction steps arranged in separate reaction zones.

The coordinates x′, y′ and z′ define a specific reaction block in an array of reaction blocks. For a four-dimensional array, x′ defines the location of a reaction block in a linear array of reaction blocks where each reaction block has a different fourth diversity introducing reaction step chemical composition introduced into the reaction zones of each reaction block with the same value of x′.

In a five-dimensional array, x′ and y′ identify a specific reaction block in a two-dimensional array of reaction blocks where the reaction blocks are preferably organized in a regular Cartesian grid arrangement with rows and columns of reaction blocks. Different fourth diversity introducing reaction step chemical compositions are introduced into the reaction zones of reaction blocks with different x′ coordinate value such that reaction blocks with a common x′ coordinate receive the same fourth chemical composition. Similarly, different fifth building block components are introduced into the reaction zones of reaction blocks with different y′ coordinate value such that reaction blocks with a common y′ coordinate value receive the same fifth chemical composition.

A six-dimensional array can be prepared by arranging two, or more preferably z′, five-dimensional combinatorial libraries of the present invention, where a different sixth building block is introduced into the reaction zone of each five-dimensional array such that each five-dimensional array is identified by a z′ coordinate value. Six coordinates, x, y, z, x′, y′ and z′, uniquely define each compound of a six-dimensional library where coordinates x′, y′ and z′ define each reaction block and coordinates x, y and z define a specific reaction zone in a three dimensional array of reaction zones in the (x′, y′, z′) reaction block of the six-dimensional library of compounds.

Other standard multiwell configurations with other dimensions are also suitable. Particularly preferred 96-well reaction blocks are Robbins Blocks and the like. Suitable multi-well reaction plates for use in the present invention are vertically stackable to form a reaction block wherein equivalent individual wells on two or more plates are aligned vertically in a column when two or more plates are stacked together. Further, the stackable plates are stacked or joined in such a manner that each column of equivalent wells from a plurality of plates form a isolated reaction zone that is insulated from other vertical reaction zones in the stacked plate system or reaction block. Vertical reaction zone isolation is effected by liquid tight seals between adjacent stacked plates. Sealing and isolating individual wells or vertical stacks of wells of adjacent stacked plates from contamination from proximal wells or vertical reactions zones is well known in combinatorial chemistry particularly in applications using standard 96 well reaction blocks such as a Robbins block. Preferred isolation and sealing methods for use in the present invention include compressible chemically resistant seals, compression seals, and the like.

In preferred applications using standard 96 well reaction plates, a Robbins Block end cap can be affixed to the bottom of a reaction block such that liquid reagents can be introduced into the z column of reaction zones. Alternatively two Robbins Block end caps can be affixed to the top and bottom of a reaction block such that the contents of the reaction block can be agitated, heated and/or pressurized with a gaseous reagent.

Each well of the plate, e.g., each reaction column, is capable of holding at least two solid support devices such as a polymer bead, a lantern or other support design. Preferred supports include Mimotope lanterns™, Mimotope crowns™, CD plugs, Irori Kans™, cellulose discs, polymeric spheres, tubes, discs or polyhedra and other similar supports that are designed to fit within a well of the multi-well plate. Preferably, supports are sized relative to the reaction column such that the vertical position of an individual support in a well or reaction column cannot change during the preparation or storage of the library in the reaction assembly, e.g., two or more supports in a reaction column cannot scramble positions in the vertical direction.

Liquids, gases or vacuum can be introduced into individual vertical reaction zones, rows or columns of vertical reaction zones or the entire reaction block using standard multiwell plate techniques. For example, liquids can be introduced into selected reaction zones including individual zones, rows, columns or the entire block by standard single or multi tip syringe pipet techniques. Liquids can also be introduced by other suitable methods that are compatible with traditional combinatorial multi-well plates.

Solid supports can comprise any material that can support one or more functionalizable groups to which the compounds of the library can be attached. Preferred solid supports include polymeric compositions, glass, ceramics, metals or metallic alloys or supports that comprise two or more of these materials. Preferred polymer solid supports include Merrifield resin (chloromethylated polystyrene), poly(acrylates), poly(methacrylates), sulfonated polystyrenes, and other functional polymers that are commonly used in solid phase synthesis of chemical compounds. Particularly preferred polymer supports include preferred polymers listed above that are crosslinked. Preferred polymer bound functionallizable groups include sulfonates, carboxylic acids, alkyl halides, alcohols, amines, sulfonyl halides, aldehydes, ketones, and the like. Specific examples of preferred solid supports include Mimotope lanterns™, Mimotope crowns™, CD plugs, Irori Kans™, cellulose discs, polymeric spheres, tubes, discs or polyhedra and other similar supports that are designed to fit within a well of the multi-well plate.

The present invention may be used in the synthesis of oligomeric as well as non-oligomeric compounds, such as polynucleotides, polypeptides, peptide-nucleic acids (PNAs), and the like, are well-known. Solid phase techniques suitable for combinatorial synthesis of non-oligomeric small molecules are also known in the art. Accordingly, these techniques and others can be used in conjunction with the methods and devices of the present invention.

Examples of resins suitable for solid-phase syntheses according to the present invention include glass, gold, or other colloidal metal particles or any of a large variety of polymer resins, typically made from cross-linked polymers, such as polystyrene, polystyrene-CHO, formylpolystyrene, acetyl polystyrene, chloroacetyl polystyrene, aminomethyl polystyrene, carboxypolystyrene, Merrifield Resin (cross-linked chloromethylated polystyrene). Other suitable resins include, but are not limited to, resins functionalized with formyl linker or indole linker, latex, cross-linked hydroxymethyl resin, 2-chlorotrityl chloride resin, trityl chloride resin, 4-benzyloxy, 2′,4′-dimethoxybenzhydrol resin, trityl alcohol resin, triphenyl methanol polystyrene resin, diphenylmethanol resin, benzhydrol resin, succinimidyl carbonate resin, p-nitrophenyl carbonate resin, imidazole carbonate resin, polyacrylamide resin, and the like. Resins such as those described above may be obtained, for example, from Aldrich Chemical Company (Milwaukee, Wis.), or from Advanced ChemTech, Inc. (Louisville, Ky.). Additional suitable resins include “ArgoGel”, a grafted polyethylene glycol-polystyrene(PEG/PS) copolymer (Argonaut Technologies, San Carlos, Calif.) and “TentaGel” (Rapp Polymere GmbH, Germany). Suitable solid support materials are formed into beads, cones, lanterns™, plugs or other appropriate scaffold shapes or morphologies. Other resins may be suitable for use in certain applications of the present invention and the use of such resins is within the scope of the present invention.

Solid support materials such as resins or other materials used with the present invention typically contain and/or are derivatized with any of a number of chemically reactive groups, which are in turn used to attach a linker (preferably a cleavable linker) to the support or resin. The linker in turn terminates in a suitable synthesis initiation site (reactive group) which is optionally protected, and which is used to attach the first building block reagent to the solid support. Examples of suitable reactive groups include alcohol, amine, hydroxyl, thiol, carboxylic acid, ester, amide, halomethyl, isocyanate, and isothiocyanate groups.

Exemplary cleavable linkers include chemically-cleavable linkers and photochemically cleavable linkers. The use of chemically cleavable and photochemically cleavable linkers is well known in the art, see for example Novabiochem 2000 catalog which is a information source regarding linkers and linker strategy. Chemically-cleavable linkers include sulfoester linkages (e.g. a thiolated tagged-molecule and a N-hydroxy-succinimidyl support, cleavable by increasing pH such as by using ammonium hydroxide), benzylhydryl or benzylamine linkages (e.g. a Knorr linker, cleavable by increasing acid concentration such as by using trifluoroacetic acid (TFA)), and disulfide linkages (e.g. a thiolated tagged-molecule and a 2-pyridyl disulfide support, such as a thiolsepharose from Sigma, cleavable with DTT (dithiothreitol)). Suitable photocleavable linkers include 6-nitroveratryloxycarbonyl (NVOC), α-methyl-6-nitroveratryl alcohol and other NVOC related linker compounds (PCT patent publication Nos. WO 90/15070 and WO 91/10092), ortho-nitrobenzyl-based linkers (C. P. Holmes et al., Journal of Organic Chemistry, (1995) v. 60, p. 2318) and phenacyl based linkers (D. Bellov et al., Chimia, (1985) v. 39, p. 317; and N. A. Abraham et al., Tetrahedron Letters, (1991) v. 32, p. 577).

### EXAMPLES

As is well known in the chemical arts the following abbreviations are used:

| | |
|---|---|
| DMSO: | dimethylsulfoxide |
| DCM: | dichloromethane |
| DMF: | N,N-dimethylformamide |
| Fmoc: | 9-flourenylmethyloxycarbonyl |
| DIC: | N,N'-diisopropylcarbodiimide |
| DMAP: | 4-N,N-dimethylamino-pyridine |
| TFA: | trifluoroacetic acid |
| PTFE: | poly(tetrafluoroethylene) |
| OAc: | acetoxy |
| AcOH: | acetic acid |
| DIEA: | diisopropylethylamine |
| PyBrOP: | Bromo-tris(pyrrolidino)-phosphonium |
| | hexafluorophosphate |
| BAL functionalized: | Backbone amide linker functionalized, i.e., |
| | 3,5-dimethoxy-4-formyl-phenoxy |
| MMT: | mono-para-methoxytrityl |
| NMP: | N-methyl pyrrolidinone |
| HOAt: | 1-hydroxy-7-azabenzotriazole |
| Boc: | tert-butyloxycarbonyl |
| TBTU: | 2-(1H-benzotriazole-1-yl)-1,1,3,3-tetramethyl- |
| | uronium tetrafluoroborate |
| NMM: | N-methylmorpholine |
| PyBOP ®: | benzotriazole-1-yl-oxytris-pyrrolidino-phosphonium |
| | hexafluorophosphate |
| NOS: | para-nitrobenzenesulfonyl |
| mCPBA: | meta-chloroperoxybenzoic acid |

### EXAMPLE 1

A 384 peptoid library of compounds arranged in a three dimensional array.

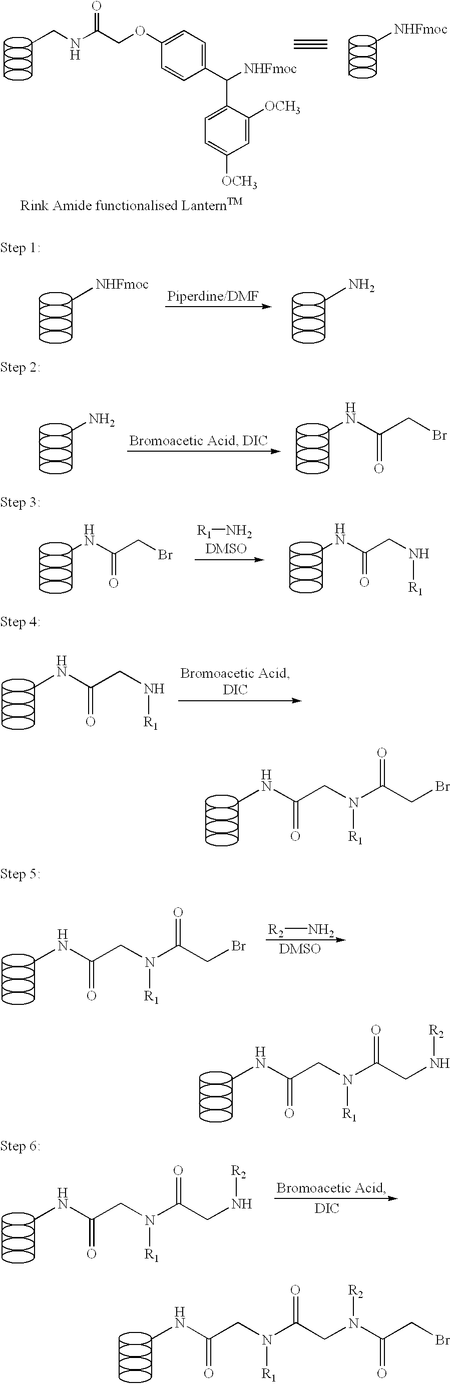

In the first step, 384 Mimotopes D-series lanterns™ pre-functionalised with Fmoc protected Rink amide linker to a loading of 0.035 mmol per lantern, were treated in a round bottomed flask with a 20% solution of piperidine in DMF (200 mL). The lanterns™, were stirred for 1 hour, then filtered and washed three times with DMF and three times with DCM. The lanterns™, were air-dried for 1 hour on the filter.

In the second step, to a suspension of the 384 lanterns™ in DMF (200 mL) was added bromoacetic acid (10 equivalents, 134.4 mmol, 18.6 g) and DIC (10 equivalents, 134.4 mmol, 16.9 g). The suspended lanterns™, stirred for 3 h and were then filtered, washed three times with DMF and three times with DCM, then air-dried on the filter for 1 h. The lanterns™, were then divided into 4 batches of 96.

In the third step, each batch of lanterns™, in separate flasks were suspended in DMSO (50 mL). To flask 1 was added 4-(trifluoromethoxy)benzylamine (15 equivalents, 50.4 mmol, 9.6 g), to flask 2 was added cyclopropylamine (15 equivalents, 50.4 mmol, 2.9 g), to flask 3 was added 2,3-dimethoxybenzylamine (15 equivalents, 50.4 mmol, 8.4 g) and to flask 4 was added 3,5-dimethylaniline (15 equivalents, 50.4 mmol, 6.1 g).The contents of the four flasks were stirred at room temperature for 16 h, then each was individually filtered and washed three times with DMF and three times with DCM then air-dried for 1 h.

In the fourth step, each of the four batches of 96 lanterns™, were suspended in DMF (50 mL) and to each was added bromoacetic acid (10 equivalents, 33.6 mmol, 4.7 g) and DIC (10 equivalents, 33.6 mmol, 4.2 g). The four reaction mixtures stirred for 3 h, and were then separately filtered, washed three times with DMF and three times with DCM. The lanterns™, were then air-dried on the filter for 1 h.

In the fifth step, the 96 lanterns™, from flask 1 were placed in the 96 wells of a 96-well filter-bottomed Robbins Flexchem™ reaction block. The 96 lanterns™, from flask 2 were then placed in the 96-wells of the same reaction block thus forming a single layer of lanterns™, on top of those from flask 1. Likewise, the lanterns™, from flask 3 were layered on top of those from flask 2, and those from flask 4 were layered on top of the layer from flask 3 such that each well in the reaction block contained 4 lanterns™. The bottom-plate was then attached to the Robbins block to seal the wells.

Stock solutions of 12 primary amines were prepared according the table below:

| | | | Quantity for 8 | |
|---|---|---|---|---|
| | | | wells = 32 | |
| Stock | | | Lanterns | |
| Solution | R₁NH₂ | FW/d | (=44.8 mm) | |
| 1 | 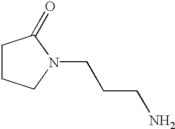 | 142.20/ 1.014 | 6.3 | ml |
| 2 | 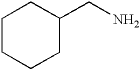 | 113.2/ 0.870 | 5.8 | ml |
| 3 | 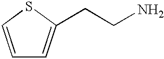 | 127.21 | 5.7 | g |
| 4 | 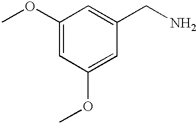 | 167.21 | 7.5 | g |
| 5 | 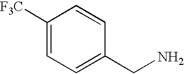 | 175.16/ 1.229 | 6.4 | ml |
| 6 | 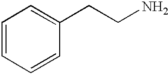 | 121.18/ 0.965 | 5.6 | ml |
| 7 | 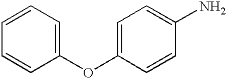 | 185.23 | 8.3 | g |
| 8 | 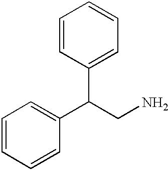 | 197.28 | 8.8 | g |
| 9 | 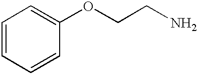 | 137.18/ 1.048 | 5.9 | ml |
| 10 | 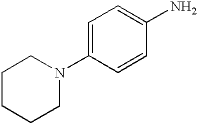 | 176.26 | 7.9 | g |
| 11 | 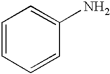 | 93.13/ 1.022 | 4.1 | ml |
| 12 | 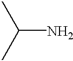 | 59.11/ 0.694 | 3.8 | ml |

The total volume of each stock solution was made up to 1 2mL by the addition of the appropriate quantity of DMSO. To each of the 8 wells in column 1 of the reaction block was added 1.5mL of stock solution 1. To each of the 8 wells in column 2 was added 1.5 mL of stock solution 2 and so on until the 8 wells in each of the 12 columns of the reaction block contained 1.5 mL of the appropriate stock solution. The top-plate of the Robbins block was then attached and the sealed block was then agitated on an orbital shaker for 3 days.

The Robbins Block was then unsealed and placed on a vacuum filter station and the plate was filtered to remove the amine solutions. Each well of the reactor block was then washed three times with DMF and four times with DCM.

In the sixth step, a stock solution was prepared by dissolving bromoacetic acid (8 equivalents, 108 mmol, 15 g) in DMF (160 mL). 1.3 mL of this solution was dispensed to each of the 96 wells of the 96-well reactor block. DIC (8 equivalents, 1.12 mmol, 0.14 g) was added to each of the 96 wells of the reactor block. The reactor block was sealed by attaching the top and bottom plates, and agitated on an orbital shaker for 3 h. The reactor block was then filtered on a vacuum filter station, and the lanterns™, were washed three times with DMF and three times with DCM.

In the seventh step, stock solutions of 8 primary amines were prepared according the table below:

| | | | For 112 wells = 48 | |
|---|---|---|---|---|
| | | | lanterns ™, | |
| Stock Solution | R₂NH₂ | FW/d | (25.2 mm) | |
| A | 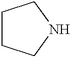 | 71.12/ 0.852 | 2.1 | ml |
| B | 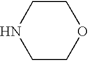 | 87.12/ 0.999 | 2.2 | ml |
| C | 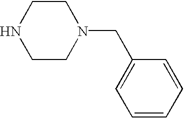 | 176.26/ 1.014 | 4.4 | ml |
| D | 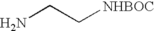 | 160 | 4.0 | g |
| E | 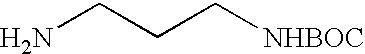 | 174 | 4.4 | g |
| F | 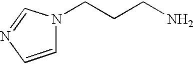 | 125.18/ 1.049 | 3.0 | ml |
| G | 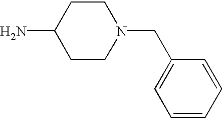 | 190.29/ 0.933 | 5.1 | ml |
| H | 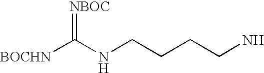 | 330 | 8.3 | g |

The total volume of each stock solution was made up to 18 mL by the addition of the appropriate quantity of DMSO. To each of the 12 wells in row A of the reaction block was added 1.5 mL of stock solution A. To each of the 12 wells in row B was added 1.5 mL of stock solution B and so on until the 12 wells in each of the 8 rows of the reaction block contained 1.5 mL of the appropriate stock solution. The top-plate of the Robbins block was then attached and the sealed block was then agitated on an orbital shaker for 16 h.

The Robbins Block was then unsealed and placed on a vacuum filter station and the plate was filtered to remove the amine solutions. Each well of the reactor block was then washed three times with DMF, once with methanol and three times with DCM.

In the eighth step, the top layer (layer 4) of 96 lanterns™, was removed from the reaction block and placed in a solid-bottomed 96 well plate (plate 4), such that the x, y coordinate of the lanterns™, in the reactor block corresponded with the x, y coordinate of the lanterns™, in the 96 well plate, e.g. the lantern from well A,1 of the reactor block was placed in position A,1 of the 96 well plate, the lantern from A,2 of the reactor block was placed in well A,2 of the 96 well plate and so on such that the lantern from well H, 12 of the reactor block is placed in well H,12 of the 96 well plate. This process is repeated, such that the lanterns™, from layer three are placed into the corresponding positions in 96 well plate 3, and the lanterns™, from layer 2 are placed in 96 well plate 2, and from layer 1 in 96 well plate 1.

Each well in the four plates was then treated with a 30% solution of TFA in DCM for 1 h to cleave the compounds from the lanterns™. The TFA/DCM solution was removed by evaporation, and the lanterns™, were removed from the wells, affording the 384 dried down compounds in the four 96 well plates.

### EXAMPLE 2

A combinatorial library of 144 (3-carboxamido-4-arylpyrrolidines) in a three dimensional array.

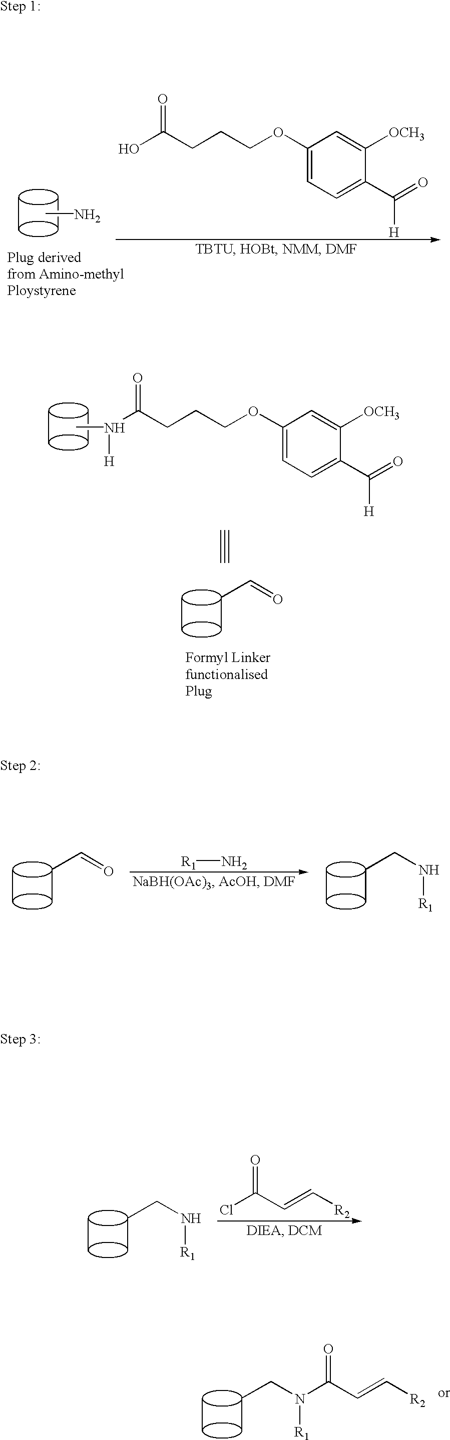

In the first step, 300 sintered aminomethyl polystyrene plugs with a loading of 75 μmmol per plug (total 22.5 mmol) were suspended in DMF (100 mL). 4-(4-Formyl-3-methoxy-phenoxy)-butyric acid (2equivalents, 45 mmol, 10.72 g), HOBt (1 equivalent, 22.5 mmol, 3.47 g) and N-methylmorpholine (4equivalents, 90 mmol, 9.9 mL) were dissolved in DMF (150 mL) and the solution stirred at 0° C. while TBTU (2 equivalents, 45 mmol, 14.45 g) was added. The solution was allowed to warm to room temperature, stirred for a further 10 mins and was then added to the suspended plugs. The plugs suspension was agitated gently for 16 h and then filtered, washed twice with DMF, three times with DCM and once with methanol. The plugs were allowed to air-dry on the filter for 1 h.

In the second step, 240 of the plugs from step 1 were divided into 6 round-bottomed flasks, each containing 40 plugs. To each flask was added DMF (95 mL) and (5 mL) followed by the amine (20 equivalents) as indicated in the table below:

| | | | For 40 Plugs |
|---|---|---|---|
| | | | 20 equivalents = |
| Flask | Amine | FW/d | 60 mmol |
| 1 | 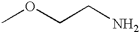 | 75.11/ 0.86 | 5.22 mL |
| 2 | 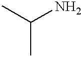 | 59.11/ 0.69 | 5.13 mL |
| 3 | 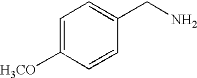 | 137.18/ 1.05 | 7.84 mL |
| 4 | 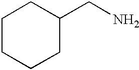 | 99.18/ 0.86 | 6.92 mL |
| 5 | 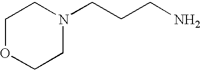 | 144.22/ 0.98 | 8.83 mL |
| 6 | 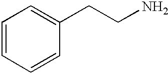 | 121.18/ 0.965 | 7.53 mL |

The suspension was gently agitated for 2 h, then sodium triacetoxyborohydride (20 equivalents, 60 mmol, 12.71 g) was added in one portion to each flask. The plugs were agitated for a further 3 h then filtered and washed once with a 10% solution of methanol in DMF, three times with DMF, once with DCM, once with methanol, once with DCM, once with methanol and once with ether. The plugs were allowed to air dry on the filter for one hour and were then dried at ambient temperature in a vacuum oven.

In the third step, 24 Varian BondElut Reservoir filter bottomed tubes were assembled in a 6 (A to F)×4 (1 to 4) array in a Janke and Kunke VX2 plastic rack. A plug from flask 1 in step 2 was added to each of the 24 tubes such that a total of 24 plugs from flask 1 were used. A plug from flask 2 in step 2 was added to each of the 24 tubes, such that a layer of plugs from step 2 was formed on top of the layer of plugs from step 1. The diameters of the tubes and the plugs are such that the layers of plugs cannot pass each other in the tubes. This process was repeated with plugs from flasks 3, 4, 5 and 6, in that order, such that 6 layers of plugs were formed in each of the 24 tubes for a total of 144 plugs.

To each of the 4 tubes in columns A, B, C and D, were added solutions of the cinnamoyl chlorides (5 equivalents, 2.25 mmol) as detailed in the table below, and diisopropylethylamine (5 equivalents, 2.25 mmol, 0.39 mL) in DCM (6 mL). To each of the 4 tubes in columns E and F were added solutions of the cinnamic acids (5 equivalents, 2.25 mmol) as detailed in the table below, DIC (5 equivalents, 2.25 mmol, 0.35 mL), diisopropylethylamine (5 equivalents, 2.25 mmol, 0.39 mL) and DMAP (2.5mol %, 0.0011 mmol, 1.4 mg), which had been allowed to stand for 10 minutes before filtering into the tubes.

| | Cinnamoyl chloride | | For 24 Plugs |
|---|---|---|---|
| | Or | | 5 equivalents = |
| Column | Cinnamic acid | FW | 9 mmol |
| A | 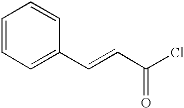 | 166.6 | 1.50 g |
| B | 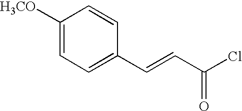 | 196.6 | 1.77 g |
| C | 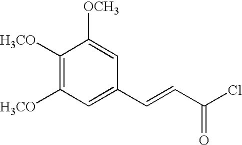 | 256.7 | 2.31 g |
| D | 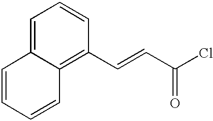 | 216.7 | 1.95 g |
| E | 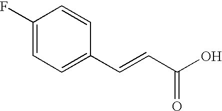 | 166.2 | 1.50 g |
| F | 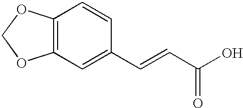 | 192.2 | 1.73 g |

Tubes in columns A to D were agitated gently for 3 h, then filtered and washed sequentially with DCM, methanol, DCM, methanol, DCM then ether. The plugs, still in their columns, were dried in a vacuum oven at ambient temperature.

Tubes in columns E and F were agitated gently for 16 h, then filtered and washed sequentially with DMF, two times with methanol, DCM, methanol, DCM, methanol and then dried in a vacuum oven at ambient temperature.

In the fourth step, to each of the 24 tubes in the array was added an equal portion of a stock solution of benzyl-(2-methoxy-ethoxymethyl)-trimethylsilanylmethyl-amine (10 equivalents, 108 mmol, 30.40 g) in DCM (120ml) and TFA (1.8 mL of a 1.0 M solution in DCM) at 0° C. The tubes were allowed to warm to room temperature, and then gently agitated for 48 h. The tubes were filtered and washed twice with DCM, methanol, DCM, methanol and finally ether. The tubes were air-dried on the filter and then dried in a vacuum oven at ambient temperature for 15 h.

In the fifth step, to a solution of diisopropylethylamine (6 equivalents, 64.5 mmol, 11.26 mL) in DCM (135 mL) at 0° C., was added 1-chloroethylchloroformate (3 equivalents, 32.4 mmol, 3.51 mL). The mixture stirred at 0° C. for 5 mins, and was then decanted in equal portions into the 24 reaction tubes containing the layers of plugs. The cartridges were agitated for 60-90 min whilst being allowed to warm to room temperature, then agitated for a further 30 min. The solution was removed by filtration, then the plugs were washed with DCM, methanol and DCM again.

A solution of methanol (28.5 mL) and acetic acid (1.5 mL) in DCM (120 mL) was prepared, and then decanted in equal portions to the 24 tubes in the array. The tubes were sealed and then agitated for 48 h at ambient temperature, then filtered and washed twice with a 5% solution of diisopropylethylamine in DCM, DCM, methanol, DCM, methanol and once with ether. The tubes were air-dried on the filter for 1 h, then dried in a vacuum oven at ambient temperature.

In the sixth step, to each of the six tubes in row 1 of the array, was added a solution of 4-isopropylbenzaldehyde (5 equivalents, 2.25 mmol, 0.34 mL) in DMF (5.7 mL) and acetic acid (0.3 mL). The plugs were gently agitated for 2 h, then sodium triacetoxyborohydride (5 equivalents, 2.25 mmol, 0.48 g) was added to each of the six tubes. These shook at ambient temperature for 16 h and were then filtered and washed with a 10% solution of methanol in DMF, followed by DMF, methanol, DCM, methanol, DCM, and methanol again before drying in a vacuum oven at ambient temperature.

To each of the six tubes in row 2 of the array, was added a solution of 4-toluoylchloride (3 equivalents, 1.35 mmol, 0.21 g) and diisopropylethylamine (5 equivalents, 2.25 mmol, 0.39 mL) in DCM (6 mL), The suspensions were agitated gently for 2 h, then filtered and washed three times with DCM, once with methanol and once with ether before air-drying in the tubes. The tubes were dried in a vacuum oven at ambient temperature for 16 h.

To each of the six tubes in row 3 of the array was added a solution of 4-toluenesulphonyl chloride (5 equivalents, 2.25 mmol, 0.43 g), diisopropylethylamine (7 equivalents, 3.15 mmol, 0.55 mL) and DMAP (0.1 equivalent, 0.045 mmol, 5.5 mg) in DCM (6 mL). The suspensions were agitated gently for 16 h, then filtered and washed three times with DCM, once with methanol and once with ether before air-drying in the tubes. The tubes were dried in a vacuum oven at ambient temperature for 16 h.

To each of the six tubes in row 4 of the array was added a solution of cyclohexylisocyanate (10 equivalents, 4.5 mmol, 0.56 g) and DMAP (0.1 equivalent, 0.045 mmol, 5.5 mg) in DCM (6 mL). The plugs were agitated gently for 16 h, then filtered and washed three times with DCM, once with methanol and once with ether before air-drying in the tubes. The tubes were dried in a vacuum oven at ambient temperature for 16 h.

In the seventh step, the top layer (layer 6) of 24 plugs was removed from the reaction block and placed in a rack of glass vials in a 6×4 array (rack 6), such that the x, y coordinate of the plugs from the reaction block corresponded with the x, y coordinate of the plugs in the array of vials, e.g. the plug from reaction tube A,1 of the reaction block was placed in position A,1 of the 24-vial rack, the plug from A,2 of the reaction block was placed in well A,2 of the 24-vial rack, and so on such that the plug from reaction tube F,4 of the reaction block is placed in well F,4 of the 24-vial rack. This process is repeated, such that the plugs from layer 5 are placed into the corresponding positions in 24-vial rack 5, and the plugs from layer 4 are placed in 24-vial rack 4, from layer 3 in 24-vial rack 3, from layer 2 in 24-vial rack 2, and from layer 1 in 24-vial rack 1.

Each vial in the 6 racks was then treated with 2 mL of a 50% solution of TFA in DCM for 2 h to cleave the compounds from the plugs. The TFA/DCM solution was removed by filtration, and the plugs were washed with methanol (2 mL) and then DCM (2 mL). The combined filtrate and washings were evaporated in vacuo affording the desired 144 compounds.

### EXAMPLE 3

A combinatorial library of 384 heteroaromatic compounds arranged in a three dimensional array.

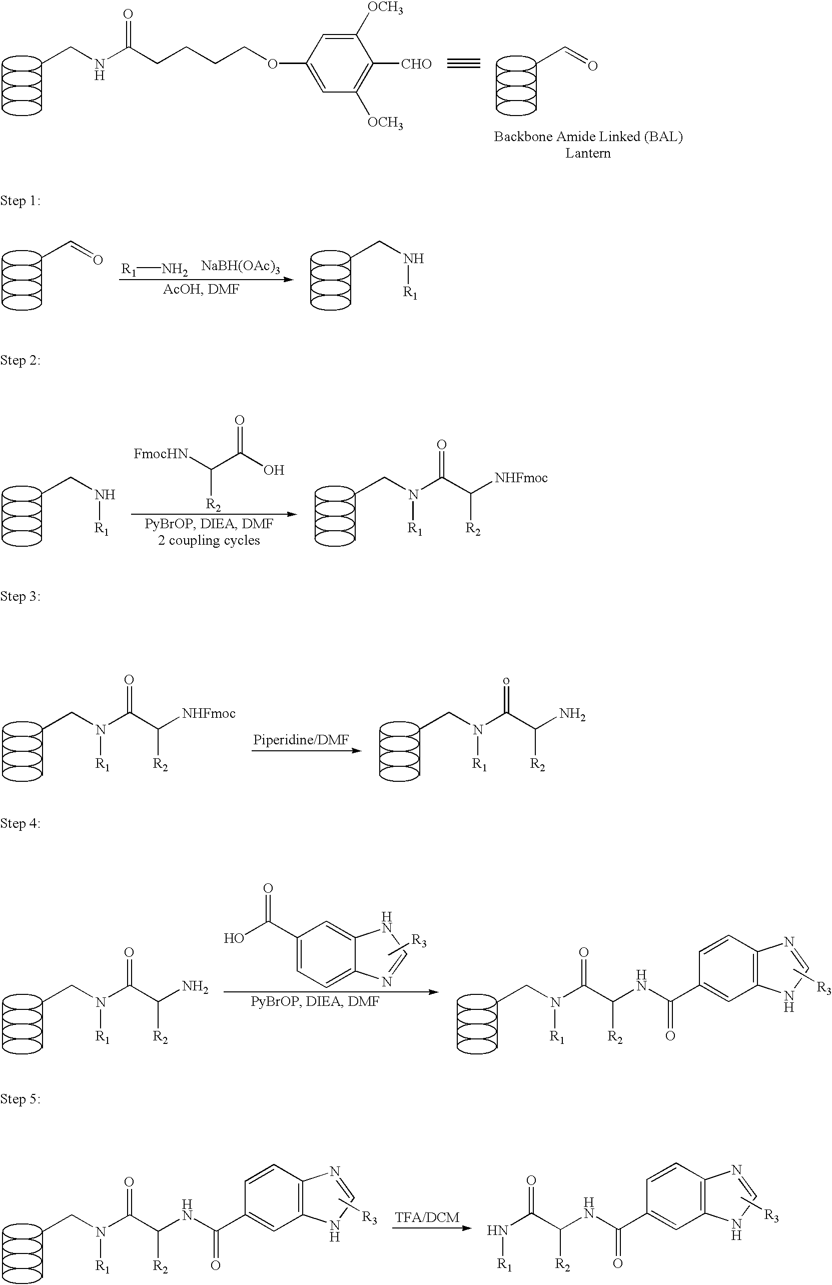

In the first step, 384 BAL functionalised D-series Lanterns™, loading=36 μmol/lantern, were divided into 4 round-bottomed flasks 1, 2, 3 and 4, each containing 96 lanterns™. To each flask was added DMF (49 mL) and acetic acid (1 mL) followed by the amine (30 equivalents) as indicated in the table below:

| | | | For 96 lanterns ™ | |
|---|---|---|---|---|
| | | | 30 equivalents = | |
| Flask | Amine R₁NH₂ | FW/d | 104 mmol | |
| 1 | 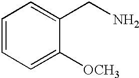 | 137.18/ 1.051 | 13.6 | mL |
| 2 | 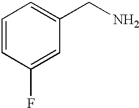 | 125.15/ 1.097 | 11.9 | mL |
| 3 | 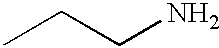 | 59.11/ 0.719 | 8.5 | mL |
| 4 | 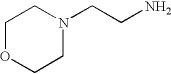 | 130.19 | 13.54 | g |

The reactions were shaken at room temperature for 1 hour. Sodium triacetoxyborohydride (10 equivalents, 34.7 mmol, 7.35 g) was added slowly to each flask, and the reactions were shaken at room temperature overnight.

Each batch of lanterns™ were filtered and washed (3×DMF, 3×MeOH, 3×DCM, MeOH, DCM, MeOH, DCM, MeOH, 2×DCM), and air-dried on the filter.

In the second step, the 96 lanterns™ from flask 1 were placed in the 96 wells of a 96-well filter-bottomed Robbins Flexchem™ reaction block. The 96 lanterns™ from flask 2 were then placed in the 96-wells of the same reaction block thus forming a single layer of lanterns™ on top of those from flask 1. Likewise, the lanterns™ from flask 3 were layered on top of those from flask 2, and those from flask 4 were layered on top of the layer from flask 3 such that each well in the reaction block contained 4 lanterns™. The bottom-plate was then attached to the Robbins block to seal the wells.

Stock solutions of 12 Fmoc-protected amino acids in DMF (12 mL) were prepared according the table below:

| | | | For 32 |
|---|---|---|---|
| | | | lanterns ™ |
| Stock | | | 3 equivalents = |
| Solution | Amino Acid | FW | 3.46 mmol |
| 1 | 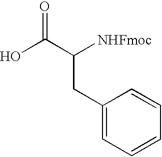 | 387.15 | 1.34 g |
| 2 | 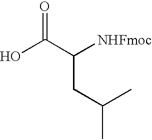 | 353.41 | 1.22 g |
| 3 | 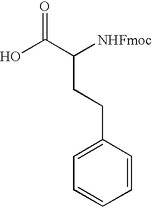 | 401.45 | 1.39 g |
| 4 | 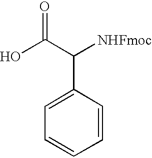 | 373.4 | 1.29 g |
| 5 | 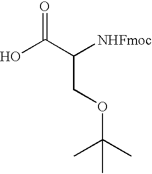 | 383.4 | 1.33 g |
| 6 | 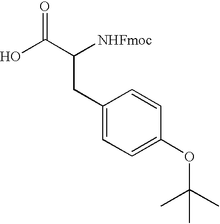 | 459.5 | 1.59 g |
| 7 | 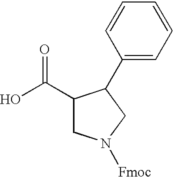 | 413.47 | 1.43 g |
| 8 | 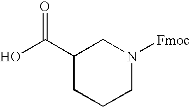 | 351.4 | 1.22 g |
| 9 | 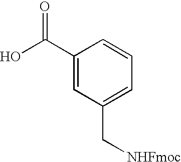 | 373.4 | 1.29 g |
| 10 | 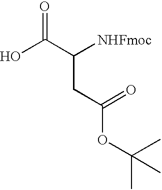 | 411.45 | 1.42 g |
| 11 | 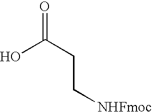 | 311.33 | 1.08 g |
| 12 | 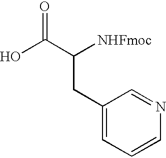 | 388.42 | 1.34 g |

To each of the 12 stock solutions was added diisopropylethylamine (6 equivalents, 6.91 mmol, 1.2 mL), and PyBrOP (3 equivalents, 3.46 mmol, 1.61 g).

To each of the 8 wells in column 1 of the reaction block was added 1.5 mL of stock solution 1. To each of the 8 wells in column 2 was added 1.5 mL of stock solution 2 and so on until the 8 wells in each of the 12 columns of the reaction block contained 1.5 mL of the appropriate stock solution. The top-plate of the Robbins block was then attached and the sealed block was-then agitated on an orbital shaker for 16 h.

The block was drained by filtration, and the lanterns™ washed (3×DMF, 3×MeOH, 3×DCM, MeOH, DCM, MeOH, DCM, MeOH, 2×DCM), and air-dried on the filter. The lanterns™ then underwent a second coupling cycle under exactly the same conditions, were filtered and washed (3×DMF, 3×MeOH, 3×DCM, MeOH, DCM, MeOH, DCM, MeOH, 2×DCM), and air-dried on the filter.

In the third step, each of the 96 wells was treated with a 20% solution of piperidine in DMF (total volume=1.5 mL). The block was closed, and the reaction was shaken at room temperature for 1 hour. The solvent was drained, and the same operation was repeated.

The block was opened and the lanterns™, were washed thoroughly (3×DMF, 3×MeOH, 3×DCM, MeOH, DCM, MeOH, DCM, MeOH, 2×DCM), and air-dried on the filter.

In the fourth step, stock solutions of 8 carboxylic acids in DMF (18 mL) were prepared according the table below:

| | | | For 48 lanterns ™ |
|---|---|---|---|
| Stock Solution | Carboxylic Acid | FW | 3 equivalents = 5.18 mmol |
| A | 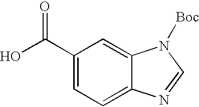 | 262.1 | 1.36 g |
| B | 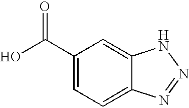 | 163.13 | 0.85 g |
| C | 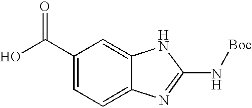 | 277.11 | 1.43 g |
| D | 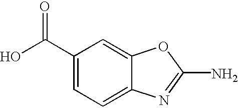 | 178.14 | 0.92 g |
| E | 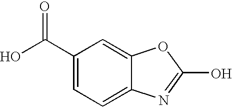 | 179.13 | 0.93 g |
| F | 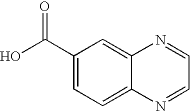 | 174.16 | 0.90 g |
| G | 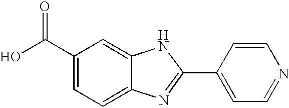 | 239.23 | 1.24 g |
| H | 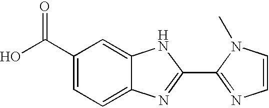 | 242.23 | 1.25 g |

To each of the 8 stock solutions was added diisopropylethylamine (6 equivalents, 10.36 mmol, 1.8 mL), and PyBrOP (3 equivalents, 5.18 mmol, 2.41 g).

To each of the 12 wells in row A of the reaction block was added 1.5 mL of stock solution A. To each of the 12 wells in row B was added 1.5 mL of stock solution B and so on until the 12 wells in each of the 8 rows of the reaction block contained 1.5 mL of the appropriate stock solution. The top-plate of the Robbins block was then attached and the sealed block was then agitated on an orbital shaker for 16 h.

The block was drained by filtration, and the lanterns™ washed (3×DMF, 3×MeOH, 3×DCM, MeOH, DCM, MeOH, DCM, MeOH, 2×DCM), and air-dried on the filter for 1 h, then in a vacuum oven at ambient temperature for 16 h.

In the fifth step, the top layer (layer 4) of 96 lanterns™ was removed from the reaction block and placed in a solid-bottomed 96 well PTFE cleavage plate (plate 4), such that the x, y coordinate of the lanterns™ in the reactor block corresponded with the x, y coordinate of the lanterns™ in the 96 well cleavage plate, e.g. the lantern from well A,1 of the reactor block was placed in position A,1 of the 96 well cleavage plate, the lantern from A,2 of the reactor block was placed in well A,2 of the 96 well cleavage plate and so on such that the lantern from well H,12 of the reactor block is placed in well H,12 of the 96 well cleavage plate. This process is repeated, such that the lanterns™ from layer three are placed into the corresponding positions in 96 well cleavage plate 3, and the lanterns from layer 2 are placed in 96 well cleavage plate 2, and from layer 1 in 96 well cleavage plate 1.

Each of the 96 wells in the four cleavage plates was then treated with a 30% solution of TFA in DCM for 1 h to cleave the compounds from the lanterns™ and to concomitantly remove acid-labile tert-butyl protecting groups from monomers 5, 6 and 10 from step 2, and from monomers A and C from step 4. The TFA/DCM solution was removed by evaporation, and the lanterns™ were rinsed with DCM, which was again removed by evaporation. The lanterns™ were removed from the wells, affording the 384 dried down compounds in the four 96 well cleavage plates.

### EXAMPLE 4

A combinatorial library of 4800 potential aspartyl protease inhibitors arranged in a four-dimensional array.

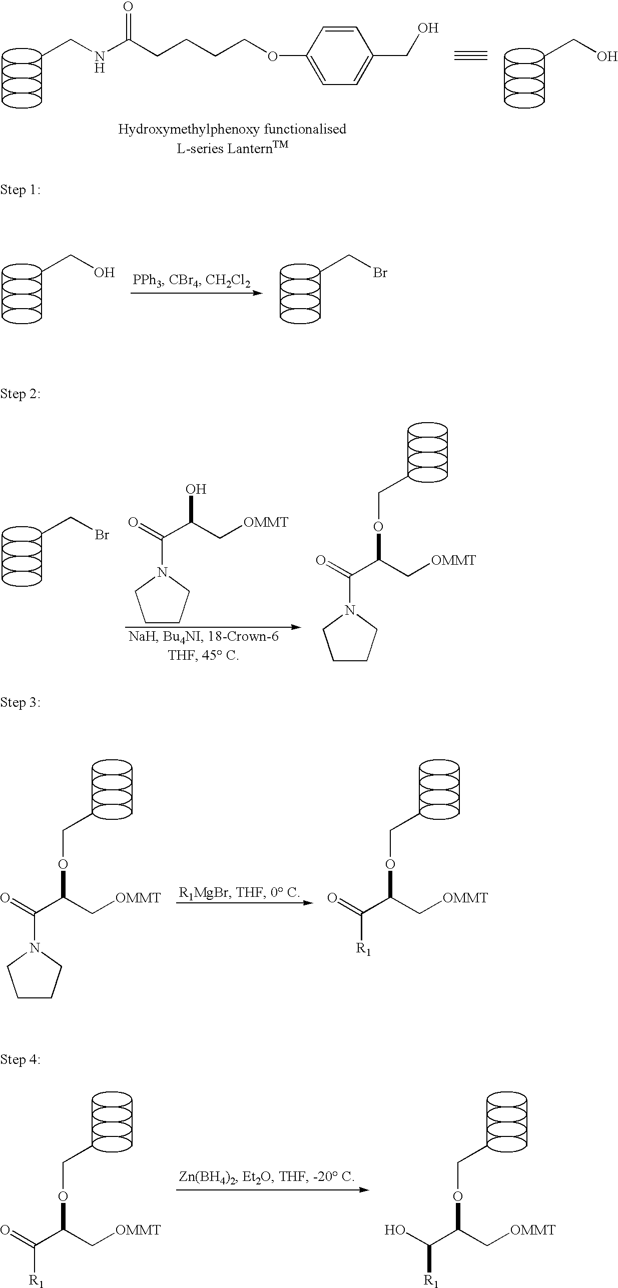

In the first step, to 4800 L-series Mimotopes Lanterns™ functionalised with the hydroxymethylphenoxy linker (loading per lantern=15 μmol, total loading=72 mmol) in DCM (700 mL) cooled to 0° C., is added carbon tetrabromide (2.5 equivalents, 180 mmol, 59.7 g) followed by a solution of triphenylphosphine (2.25 equivalents, 162 mmol, 42.4 g) in DCM (129 mL). The reaction mixture is stirred at room temperature for 3 h, then washed with DCM (×6) and dried in a vacuum oven at room temperature.

In the second step, to a suspension of NaH (3 equivalents, 216 mmol, 5.2 g) in THF (600 mL), at room temperature, is added a solution of (2S)-2-hydroxy-3-mono-p-methoxytrityl-N,N-tetramethylenepropanamide (3 equivalents, 216 mmol, 93 g) in THF (270 mL). The suspension is stirred for 1 h followed by the addition of tetrabutylammonium iodide (0.3equivalents, 21.6 mmol, 8 g), 18-crown-6 (0.3mol %, 0.216 mmol, 0.06 g) and the 4800 lanterns™ from step 1. The reaction mixture is heated to 45° C. and stirred for 2 h, then cooled to room temperature and filtered. The lanterns™, are washed with THF, THF:H₂O (2:1, ×3), THF:H₂O (1:1, ×3), THF:H₂O (1:2, ×3), THF:H₂O (2:1, ×3), THF, DMF, DCM, and MeOH. The lanterns™ are dried under vacuum.

In the third step, the lanterns™ are divided equally into 10 round-bottomed flasks such that each flask contains 480 lanterns™. The lanterns™ are suspended in THF (160 mL) at 0° C. and the appropriate Grignard reagent (5equivalents) is added according to the table below:

| | | | For 480 lanterns ™ |
|---|---|---|---|
| | | | 5 equivalents = |
| Flask | Grignard Reagent | FW | 36 mmol |
| 1 | 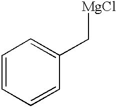 | 150.9 | 5.43 g |
| 2 | 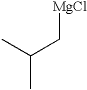 | 116.9 | 4.21 g |
| 3 | 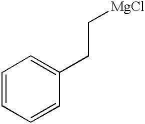 | 164.9 | 5.94 g |
| 4 | CH₃MgCl | 74.8 | 2.69 g |
| 5 | 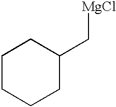 | 156.9 | 5.65 g |
| 6 | 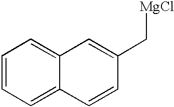 | 200.9 | 7.23 g |
| 7 | 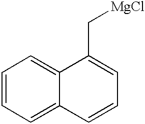 | 200.9 | 7.23 g |
| 8 | 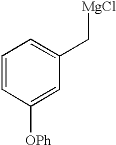 | 242.98 | 8.75 g |
| 9 | 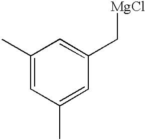 | 178.9 | 6.44 g |
| 10 | 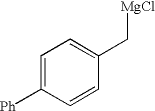 | 227.0 | 8.17 g |

The reaction mixtures are stirred at 4° C. for 20 h, then filtered separately and the batches of lanterns™ washed with THF, acetone (×3), 0.28M hydrocinnamic acid in THF (×3), DMF (×3) and DCM (×3), then dried in a vacuum oven at ambient temperature.

In the fourth step, to the 10 separate batches of lanterns™ stirring in THF (160 mL) at −20° C., is added zinc borohydride (5 equivalents, 36 mmol, 90 mL of a 0.4M solution in ether). The reaction is stirred at −20° C. for 20 h and is then allowed to warm to 0° C. over 1.5 h. The batches of lanterns™ are filtered separately, and washed with THF, ethanolamine:H₂O:THF (10:2:88 by volume, ×3), DMF (×3) and DCM (×3). The lanterns™ are then dried in a vacuum oven at room temperature.

In the fifth step, to the 10 separate batches of lanterns™ in chloroform (200 mL) is added 4-pyrrolidinopyridine (5 equivalents, 36 mmol, 5.34 g) and 4-nitrobenzenesulphonyl chloride (3 equivalents, 21.6 mmol, 4.80 g). The lanterns™ are agitated gently at room temperature for 9 h, then filtered and washed with DCM (×5) and DMF (×4).

The batches of lanterns™ are then resuspended in DMF (200 mL) and sodium azide (10 equivalents, 72 mmol, 4.68 g) is added. The reaction mixture is stirred at 45° C. for 24 h, then filtered and washed with DMF (×2), DMF:H₂O(1:1, ×3), DMF (×2) and DCM (×5) and then air-dried on the filter.

In the sixth step, the separate batches of lanterns™ are suspended in DCM (160 mL) and are treated with a 1% solution of 4-toluenesulphonic acid in DCM (total volume per batch=200 mL) for 1 h. The lanterns™ are filtered and the process repeated three times. The lanterns™ are filtered and washed with a 3% solution of methanol in DCM (×3), and DCM (×5). The batches of lanterns™ are then resuspended in chloroform (200 mL) and treated with pyridine (5 equivalents, 36 mmol, 2.85 g) and 4-nitrobenzenesulphonyl chloride (3 equivalents, 21.6 mmol, 4.80 g). The reaction mixtures are agitated at room temperature for 9 h, then filtered and washed with DCM (×2), DMF (×3) and DCM (×5). The Lanterns™ are then dried in a vacuum oven at room temperature.

In the seventh step, 96 of the lanterns™ from flask 1 are placed in the 96 wells of a 96-well filter-bottomed Robbins Flexchem™ reaction block labelled reaction block 1. This process is repeated with the remaining 384 lanterns™ from flask 1 which are placed individually into the wells of four more 96-well Robbins Flexchem™ reaction blocks labelled reaction blocks 2, 3, 4 and 5 respectively. 96 of the lanterns™ from flask 2 are then placed in the 96-wells of reaction block 1, thus forming a single layer of lanterns™ on top of those from flask 1. This process is repeated with the remaining 384 lanterns™ from flask 2, which are layered on top of the lanterns™ from flask 1 in the remaining 4 reaction blocks. Likewise, the 480 lanterns™ from flask 3 are layered on top of those from flask 2 in the five reaction blocks, and those from flask 4 were layered on top of the layers from flask 3, and so on until all of the 4800 lanterns™ from the 10 round-bottomed flasks form 10 layers in each of the 5 reaction blocks. The bottom-plates are then attached to the Robbins blocks to seal the wells.

Stock solutions of 12 primary amines (10 equivalents) in NMP (60 mL) were prepared according the table below:

| | | | Quantity for | |
|---|---|---|---|---|
| | | | 40 wells = 400 | |
| | | | Lanterns ™ 10 | |
| Stock | | | equivalents = | |
| Solution | Amine | FW/d | 60 mmol | |
| 1 | 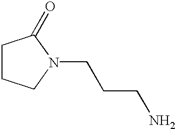 | 142.20/ 1.014 | 8.4 | ml |
| 2 | 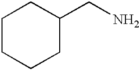 | 113.2/ 0.870 | 7.8 | ml |
| 3 | 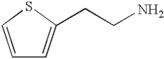 | 127.21 | 7.6 | g |
| 4 | 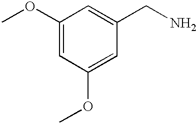 | 167.21 | 10.0 | g |
| 5 | 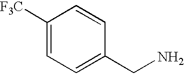 | 175.16/ 1.229 | 8.6 | ml |
| 6 | 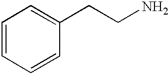 | 121.18/ 0.965 | 7.53 | ml |
| 7 | 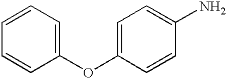 | 185.23 | 11.11 | g |
| 8 | 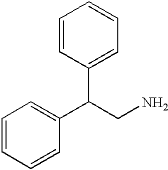 | 197.28 | 11.84 | g |
| 9 | 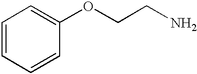 | 137.18/ 1.048 | 7.85 | ml |
| 10 | 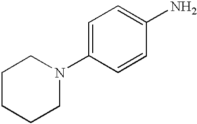 | 176.26 | 10.58 | g |
| 11 | 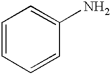 | 93.13/ 1.022 | 5.47 | ml |
| 12 | 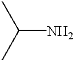 | 59.11/ 0.694 | 5.11 | ml |

1.5 mL of stock solution 1 was added to each of the 8 wells in column 1 of each of reaction blocks 1 to 5. 1.5 mL of stock solution 2 was added to each of the 8 wells in column 2 of each of reaction blocks 1 to 5. The process is repeated for the remaining 10 stock solutions, such that 1.5 mL of appropriate stock solution is placed into each well in the appropriate columns in the five reaction blocks.

The reaction blocks are then sealed by attaching the top and bottom plates. The blocks are then gently rotated in an oven at 80° C. for 36 h. After cooling to ambient temperature, the top and bottom plates are removed, and the wells are filtered under vacuum. Each well in the 5 reaction blocks is washed with NMP (×3), THF (×2), DCM (×3) and ether, then dried in a vacuum oven at room temperature for 16 h.

In the eighth step, stock solutions of 8 carboxylic acids in NMP (45 mL) are prepared according the table below:

| | | | For 600 | |
|---|---|---|---|---|
| | | | Lanterns 4 | |
| | | | equiva- | |
| Stock | | | lents = | |
| Solution | Carboxylic acid | FW | 36 mmol | |
| A | 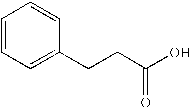 | 150.17 | 5.41 | g |
| B | 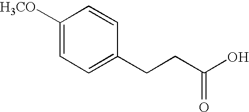 | 180.2 | 6.49 | g |
| C | 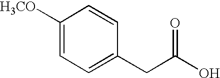 | 166.17 | 5.98 | g |
| D | 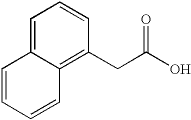 | 186.21 | 6.70 | g |
| E | 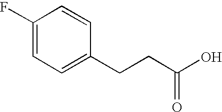 | 168.06 | 6.1 | g |
| F | 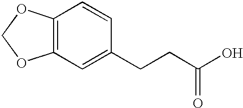 | 194.06 | 6.99 | g |
| G | 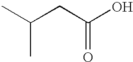 | 102.07 | 3.67 | g |
| H | 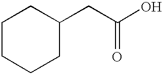 | 142.2 | 5.12 | g |

To each stock solution is added PyBOP (4 equivalents, 36 mmol, 18.73 g), HOAt (4 equivalents, 36 mmol, 4.90 g) and DIEA (12 equivalents, 108 mmol, 18.81 mL).

The volumes of the stock solutions are then made up to 90 mL by addition of NMP.

1.5 mL of stock solution A was added to each of the 12 wells in row A of each of reaction blocks 1 to 5. 1.5 mL of stock solution B was added to each of the 12 wells in row B of each of reaction blocks 1 to 5. The process is repeated for the remaining 6 stock solutions, such that 1.5 mL of appropriate stock solution is placed into each well in the appropriate rows in the five reaction blocks.

The reaction blocks are then sealed by attaching the top and bottom plates. The blocks are then gently agitated at room temperature for 16 h. The top and bottom plates are removed, and the wells are filtered under vacuum. Each well in the 5 reaction blocks is washed with NMP (×3), THF (×2), DCM (×3) then dried in a vacuum oven at room temperature for 16 h.

In the ninth step, a stock solution of tin(II) chloride (30.34 g), thiophenol (70.52 g) and triethylamine (111.5 mL) in sufficient volume of THF such that the overall volume of the solution is 800 mL, is prepared. To each of the 480 wells in the 5 reaction blocks is added 1.5 mL of the stock solution. The reaction blocks are sealed by the addition of the top and bottom plates, and then agitated for 4 h at room temperature. The lanterns™ are then filtered and washed with THF:H₂O (1:1), THF (×3), and DCM (×3). The lanterns™ are then dried in a vacuum oven at ambient temperature for 16 h.

In the tenth step, stock solutions of 5 carboxylic acids (4 equivalents) are prepared according to the table below:

| | | | For 960 Lanterns | |
|---|---|---|---|---|
| Stock | | | 4 equivalents = | |
| Solution | Carboxylic acid | FW | 57.6 mmol | |
| 1 | 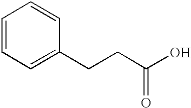 | 150.17 | 8.65 | g |
| 2 | 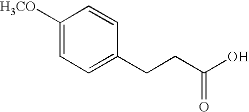 | 180.2 | 10.38 | g |
| 3 | 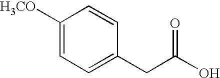 | 166.17 | 9.57 | g |
| 4 | 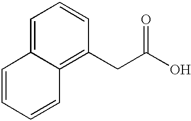 | 186.21 | 10.72 | g |
| 5 | 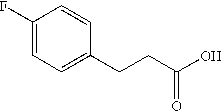 | 168.06 | 9.68 | g |

To each stock solution is added PyBOP (4 equivalents, 57.6 mmol, 29.97 g), HOAt (4 equivalents, 57.6 mmol, 7.95 g) and DIEA (12 equivalents, 172.8 mmol, 30.04 mL).

The volumes of the stock solutions are then made up to 144 mL by addition of NMP. 1.5 mL of stock solution 1 was added to each of the 96 wells in reaction block 1. 1.5 mL of stock solution 2 was added to each of the 96 wells in reaction block 2. The process is repeated for the remaining 3 stock solutions, such that 1.5 mL of appropriate stock solution is placed into each of the wells in the remaining reaction blocks.

The reaction blocks are then sealed by attaching the top and bottom plates. The blocks are then gently agitated at room temperature for 16 h. The top and bottom plates are removed, and the wells are filtered under vacuum. Each well in the 5 reaction blocks is washed with NMP (×3), THF (×2), DCM (×3) then dried in a vacuum oven at room temperature for 16 h.

In the eleventh step, the top layer (layer 10) of 96 lanterns™ is removed from the reaction block 1 and placed in a solid-bottomed 96 well PTFE cleavage plate (plate 1-10), such that the x, y coordinate of the lanterns™ in the reaction block corresponds with the x, y coordinate of the lanterns™ in the 96 well cleavage plate, e.g. the lantern from well A,1 of the reaction block is placed in position A,1 of the 96 well cleavage plate, the lantern from A,2 of the reaction block is placed in well A,2 of the 96 well cleavage plate and so on such that the lantern from well H,12 of the reaction block is placed in well H,12 of the 96 well cleavage plate. This process is repeated, such that the lanterns™ from layer 9 are placed into the corresponding positions in 96 well cleavage plate (plate 1-9), and the lanterns™ from layer 8 are placed in 96 well cleavage plate 1-8, and so on until all ten layers from reaction block 1 are placed in to the corresponding wells of the 10 corresponding 96-well cleavage blocks.

This process is repeated for each of the 10 layers from the remaining 4 reaction blocks to generate a total of 50 96-well cleavage plates.

Each of the 96 wells in the 50 cleavage plates is then treated with a 30% solution of TFA in DCM for 1 h to cleave the compounds from the lanterns™. The TFA/DCM solution was removed by evaporation, and the lanterns™ were rinsed with DCM, which was again removed by evaporation. The lanterns™ were removed from the wells, affording the 4800 dried down compounds in the 50 96 well cleavage plates.

### EXAMPLE 5

Preparation of a 5760 membered pyrimidine library in a five dimensional array.

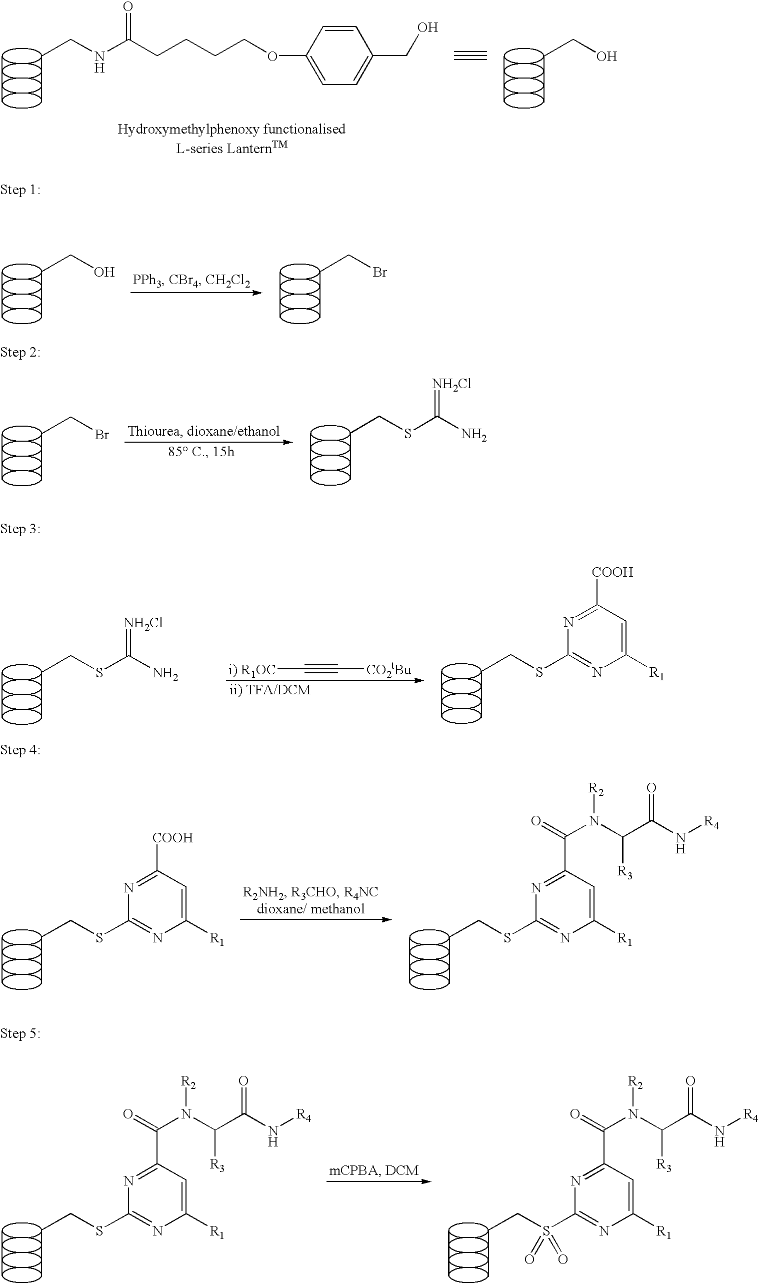

In the first step, to 5760 L-series Mimotopes Lanterns™ functionalised with the hydroxymethylphenoxy linker (loading per lantern=15 μmol, total loading=86.4 mmol) in DCM (800 mL) cooled to 0° C., is added carbon tetrabromide (2.5 equivalents, 216 mmol, 71.6 g) followed by a solution of triphenylphosphine (2.25 equivalents, 194.4 mmol, 50.9 g) in DCM (155 mL). The reaction mixture is stirred at room temperature for 3 h, then washed with DCM (×6) and dried in a vacuum oven at room temperature.

In the second step, to the 5760 lanterns™ in a 4:1 solution of dioxane/ethanol (900 mL) is added thiourea (5 equivalents, 432 mmol, 32.9 g). The suspension is heated to 85° C. and agitated for 15 h, and then washed with ethanol at 70° C. (×4), dioxane (×2), and pentane (×2). The lanterns™, are then air-dried, and then dried in a vacuum oven at 60° C. for 16 h. In the third step, the lanterns™ are divided equally into 5 round-bottomed flasks (flasks 1-5) such that each flask contains 1152 lanterns™. The lanterns™ are suspended in DMF (200 mL) at room temperature and the appropriate acetylenic ketone (1.2 equivalents) is added according to the table below:

| | | | For 1152 lanterns ™ | |
|---|---|---|---|---|
| | | | 1.2 equivalents = | |
| Flask | Acetylenic Ketone | FW | 20.7 mmol | |
| 1 | 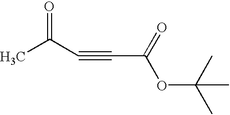 | 168.19 | 3.48 | g |
| 2 | 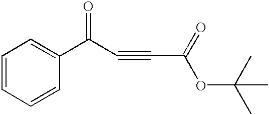 | 230.3 | 4.77 | g |
| 3 | 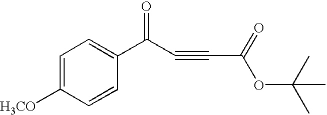 | 260.1 | 5.4 | g |
| 4 | 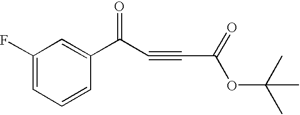 | 248.25 | 5.1 | g |
| 5 | 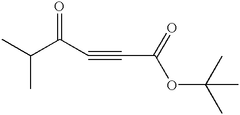 | 196.24 | 4.1 | g |

Each flask is then equipped with a syringe pump and a solution of diisopropylethylamine (1.5 equivalents, 25.9 mmol, 3.34 g) in dioxane (50 mL) is added slowly over a period of 24 h as the flasks are agitated.

The flasks are then vortexed for an additional 24 h, then filtered and washed with DMF (×3), isopropanol (×3), dioxane (×3), isopropanol (×3), DCM (×3) and pentane (×3).

The lanterns™ are then resuspended in DCM (300 mL) and treated with TFA (300 mL) at room temperature for 20 min. The lanterns™ are then filtered and washed with DCM (×4), DCM:Et₃N (4:1, ×3), DMF (×2), isopropanol (×2), dioxane/2N HCl (×3), DMF (×2), isopropanol (×2) and pentane (×2). The lanterns™, are then dried in a vacuum oven at 50° C. for 16 h.

In the fourth step, 96 of the lanterns™ from flask 1 are placed in the 96 wells of a 96-well filter-bottomed Robbins Flexchem™ reaction block labelled reaction block 1. Then another layer of 96 lanterns from flask 1 are formed on top of the first layer. This process is repeated with the remaining 960 lanterns™ from flask 1 which are placed into the wells of five more 96-well Robbins Flexchem™ reaction blocks labelled reaction blocks 2, 3, 4, 5 and 6 respectively, such that each reaction block contains two layers of lanterns from flask 1. 192 of the lanterns™ from flask 2 are then placed in the 96-wells of reaction block 1, thus forming a double layer of lanterns™ on top of the double layer from flask 1. This process is repeated with the remaining 960 lanterns™ from flask 2, which are layered on top of the lanterns™ from flask 1 in the remaining 5 reaction blocks. Likewise, the 1152 lanterns™ from flask 3 are layered on top of those from flask 2 in the five reaction blocks, and those from flask 4 were layered on top of the layers from flask 3, and so on until all of the 5760 lanterns™ from the 5 round-bottomed flasks form 10 layers in each of the 6 reaction blocks. The bottom-plates are then attached to the Robbins blocks to seal the wells.

Stock solutions of 8 primary amines (5 equivalents) in dioxane/methanol (4:1, 36 mL) are prepared according the table below:

| | | | For 720 lanterns ™ | |
|---|---|---|---|---|
| | | | 5 equivalents = | |
| Flask | Amine | FW | 54 mmol | |
| A | 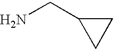 | 71.12 | 3.84 | g |
| B | 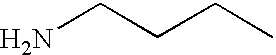 | 73.14 | 3.95 | g |
| C | 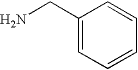 | 107.15 | 5.79 | g |
| D | 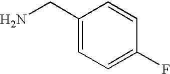 | 125.14 | 6.76 | g |
| E | 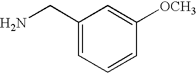 | 137.18 | 7.41 | g |
| F | 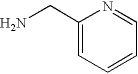 | 108.14 | 5.84 | g |
| G | 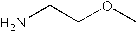 | 75.11 | 4.06 | g |
| H | 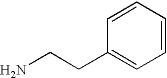 | 121.18 | 6.54 | g |

0.5 mL of stock solution A was added to each of the 12 wells in rows A of each of reaction blocks 1 to 6. 0.5 mL of stock solution B was added to each of the 12 wells in rows B of each of reaction blocks 1 to 6. The process is repeated for the remaining 6 stock solutions, such that 0.5 mL of appropriate stock solution is placed into each well in the appropriate rows in the six reaction blocks.

Stock solutions of 12 aldehydes (5 equivalents) in dioxane/methanol (4:1, 24 mL) are prepared according the table below:

| | | | For 480 lanterns ™ | |
|---|---|---|---|---|
| | | | 5 equivalents = | |
| Flask | Aldehyde | FW | 36 mmol | |
| 1 | 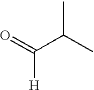 | 72.11 | 2.60 | g |
| 2 | 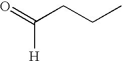 | 72.11 | 2.60 | g |
| 3 | 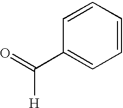 | 106.12 | 3.82 | g |
| 4 | 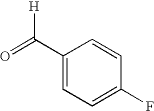 | 124.11 | 4.47 | g |
| 5 | 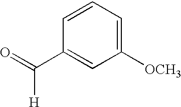 | 136.15 | 4.90 | g |
| 6 | 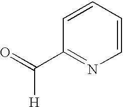 | 107.11 | 3.86 | g |
| 7 | 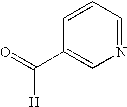 | 107.11 | 3.86 | g |
| 8 | 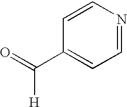 | 107.11 | 3.86 | g |
| 9 | 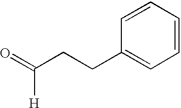 | 134.18 | 4.83 | g |
| 10 | 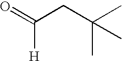 | 100.16 | 3.61 | g |
| 11 | 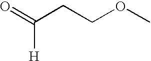 | 88.11 | 3.17 | g |
| 12 | 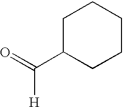 | 112.17 | 4.04 | g |

0.5 mL of stock solution 1 is added to each of the 8 wells in column 1 of each of reaction blocks 1 to 6. 0.5 mL of stock solution 2 is added to each of the 8 wells in column 2 of each of reaction blocks 1 to 6. The process is repeated for the remaining stock solutions, such that 0.5 mL of appropriate stock solution is placed into each well in the appropriate columns in the six reaction blocks.

Stock solutions of 6 isonitriles (5 equivalents) in dioxane/methanol (4:1, 48 mL) are prepared according the table below:

| | | | For 960 lanterns ™ | |
|---|---|---|---|---|
| | | | 5 equivalents = | |
| Flask | Isonitrile | FW | 72 mmol | |
| 1 | 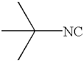 | 83.13 | 5.99 | g |
| 2 | 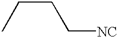 | 83.13 | 5.99 | g |
| 3 | 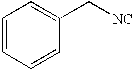 | 117.15 | 8.43 | g |
| 4 | 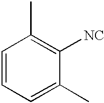 | 131.17 | 9.44 | g |
| 5 | 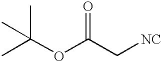 | 141.17 | 10.16 | g |
| 6 | 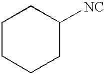 | 109.17 | 7.86 | g |

0.5 mL of stock solution 1 is added to each of the 96 wells of reaction block 2. The process is repeated for the remaining 4 stock solutions and the remaining 4 reaction blocks, such that the 96 wells of each reaction block are filled with the appropriate stock solution.

The 6 reaction blocks are then sealed top and bottom, and then agitated with heating at 75° C. for 72 h. The wells are then filtered and the lanterns™ washed with dioxane (×3), DMF (×3), DCM (×3), isopropanol (×3), DCM (×3) and pentane (×2). The lanterns™, are then air-dried, and then dried in a vacuum oven at 50° C. for 16 h. In the fifth step, a stock solution of mCPBA (3 equivalents, 259.2 mmol, 44.73 g) in DCM (864 mL) is prepared. To each of the 96 wells of the 6 reaction blocks is added 1.5 mL of the stock solution. The wells are then sealed and the reaction blocks agitated at room temperature for 16 h, then filtered and washed with DCM (×3), isopropanol (×3), and pentane (×2), then dried in a vacuum oven at ambient temperature.

In the sixth step, the top layer (layer 10) of 96 lanterns™ is removed from the reaction block 1 and placed in a solid-bottomed 96 well PTFE cleavage plate (plate 1-5), such that the x, y coordinate of the lanterns™ in the reaction block corresponds with the x, y coordinate of the lanterns™ in the 96 well cleavage plate, e.g. the lantern from well A,1 of the reaction block is placed in position A,1 of the 96 well cleavage plate, the lantern from A,2 of the reaction block is placed in well A,2 of the 96 well cleavage plate and so on such that the lantern from well H,12 of the reaction block is placed in well H,12 of the 96 well cleavage plate. To each well in the 96-well cleavage plate is added pyrrolidine (1 equivalent, 1 mL of a stock solution consisting of 4.02 mL pyrrolidine in 2880 mL dioxane). The lanterns™ from layer 9 of reaction block 1 are placed into the corresponding positions in a 96 well cleavage plate (plate 2-5). To each well in the 96-well cleavage plate is added piperidine (1 equivalent, 1 mL of a stock solution consisting of 4.28 mL piperidine in 2880 mL dioxane).

Layers 8 and 7 are likewise placed into 96 well cleavage plates (1-4 and 2-4), such that each lanterns'™ x, y coordinate in the cleavage plate corresponds to its x, y coordinate in the reaction block. Each of the wells in the cleavage plate containing lanterns™ from layer 8 of reaction block 1 are treated with 1 mL of the pyrrolidine stock solution. The cleavage plate containing lanterns™, from layer 7 are treated with the piperidine stock solution.

The remainder of reaction block 1 is plated out similarly, even-numbered layers being placed in cleavage plates and treated with pyrrolidine, and odd-numbered layers being placed in cleavage plates and treated with piperidine. Each of reaction blocks 2 to 6 are likewise plated out and the even-numbered layers cleaved with pyrrolidine, and the odd-numbered layers being treated with piperidine. This process generates 60 plates, 30 containing pyrrolidine stock solution and the remainder containing piperidine stock solution. The plates are then sealed and agitated for 6 h. The solutions are then evaporated under vacuum, DCM (1 mL) added to each well, and then evaporated again under vacuum. The lanterns™ were removed from the wells, affording the 5760 dried down compounds in the 60 96-well cleavage plates.

Although a preferred embodiment of the invention has been described using specific terms, such description is for illustrative purposes only, and it is to be understood that changes and variations may be made without departing from the spirit or scope of the following claims.

What is claimed is:

1. A method for preparing a spatially separated library of compounds, comprising:
providing a reaction assembly comprising a plurality of reaction zones where each reaction zone can be defined by four or more coordinates;
contacting each reaction zones with at least one chemical composition.

2. A method of claim 1 wherein a solid support is introduced into a reaction zone.

3. A method of claim 1 wherein a solid support is introduced into each reaction zone.

4. A method of claim 1 wherein at least two chemical compositions are introduced into a reaction zone.

5. A method for preparing a spatially separated combinatorial library of compounds, comprising:
a) providing a reaction assembly that comprises a plurality of reaction zones such that each reaction zone of the reaction assembly is uniquely identifiable with four or more coordinates;
b) contacting a plurality of batches of solid supports with a chemical composition in a first diversity introducing reaction step such that each batch is contacted with a different chemical composition under conditions conducive to the reaction of the chemical composition with the solid support;
c) introducing at least one solid support from step b) into each reaction zone of the reaction assembly so that reaction zones with a common z coordinate value comprise a solid support with a common reaction history from step b) and reaction zones with a different z coordinate value comprise solid supports with a different reaction history from step b);
d) contacting the solid supports contained in reaction zones with a common non-z axis coordinate value with a common chemical composition and introducing different chemical compositions into reaction zones with different non-z axis coordinate values under conditions conducive to the second diversity introducing reaction step; and
e) repeating step d) for each additional diversity introducing reaction steps such that a common chemical composition for each diversity introducing reaction step is introduced into reaction zones with a common non-z axis coordinate value such that the non-z axis is different from the non-z axes corresponding to completed diversity introducing reaction steps such that the solid support(s) of each reaction zone of the reaction assembly has a reaction history that corresponds to the coordinate values of the reaction zone.

6. A method according to claim 5, wherein there are about 4 to about 8 diversity introducing reaction steps.

7. A method of claim 5, wherein each reaction zone is uniquely identifiable by six coordinates and the library of compounds is prepared by contacting each solid support with a different combination of chemical compositions in at least six diversity introducing reaction steps.

8. A method of claim 7 for preparing a library of compounds comprising:
contacting a plurality of batches of solid supports with different chemical compositions of the first diversity introducing reaction step under conditions conducive for the reaction of the solid support with the chemical composition of the first diversity introducing reaction step such that there are Z batches of solid supports for Z different chemical compositions of the first diversity introducing reaction step and there are sufficient solid supports in each batch so that at least one solid support from a batch can be introduced into each reaction zone of the reaction assembly with a common z coordinate;
distributing solid supports to reaction zones of the reaction assembly such that reaction zones with a common z coordinate each receive a solid support from the same batch;
contacting the reaction zones of the reaction assembly with X different chemical compositions of a second diversity introducing reaction step such that all reaction zones of the reaction assembly having a common x coordinate receive the same chemical composition of the second diversity introducing reaction step under conditions conducive to reaction of the solid support with the contacting chemical composition;
contacting the reaction zones of the reaction assembly with Y different chemical compositions of a third diversity introducing reaction step such that all reaction zones of the reaction assembly having a common y coordinate receive the same chemical composition of the third diversity introducing reaction step under conditions conducive to reaction of the solid support with the contacting chemical composition;
contacting the reaction zones of the reaction assembly with X′ different chemical composition of a fourth diversity introducing reaction step such that all reaction zones of the reaction assembly having a common x′ coordinate receive the same chemical composition of the fourth diversity introducing reaction step under conditions conducive to reaction of the solid support with the contacting chemical composition;
contacting the reaction zones of the reaction assembly with Y′ different chemical composition of a fifth diversity introducing reaction step such that all reaction zones of the reaction assembly having a common y′ coordinate receive the same chemical composition of the fifth diversity introducing reaction step under conditions conducive to reaction of the solid support with the contacting chemical composition; and
contacting the solid supports of the reaction assembly with Z′ different chemical compositions of a sixth diversity introducing reaction step such that all reaction zones of the reaction assembly having a common z′ coordinate receive the same chemical composition of the sixth diversity introducing reaction step under conditions conducive to reaction of the solid support with the contacting chemical composition such that a library of compounds is prepared where the solid support(s) of each reaction zone have a different reaction history and each compound of the library can be identified by six coordinates.

9. A method of claim 5, wherein each reaction zone is uniquely identifiable by five coordinates and the library of compounds is prepared by contacting each reaction zone of the library with a different combination of chemical compositions in at least five diversity introducing reaction steps.

10. A method of claim 9 for preparing a library of compounds comprising:
contacting a plurality of batches of solid supports with different chemical compositions of a first diversity introducing reaction step under conditions conducive for a reaction of the solid support with the chemical composition of the first diversity introducing reaction step such that there are Z batches of solid supports for Z different chemical compositions of the first diversity introducing reaction step and there are sufficient solid supports in each batch so that at least one solid support from a batch can be introduced into each reaction zone of the reaction assembly with a common z coordinate;
distributing solid supports to reaction zones of the reaction assembly such that reaction zones with a common z coordinate each receive a solid support from the same batch;
contacting the reaction zones of the reaction assembly with X different chemical compositions of a second diversity introducing reaction step such that all reaction zones of the reaction assembly having a common x coordinate receive a same chemical composition of the second diversity introducing reaction step under conditions conducive to reaction of the solid support with the contacting chemical composition;
contacting the reaction zones of the reaction assembly with Y different chemical compositions of a third diversity introducing reaction step such that all reaction zones of the reaction assembly having a common y coordinate receive the same chemical composition of the third diversity introducing reaction step under conditions conducive to reaction of the solid support with the contacting chemical composition;
contacting the reaction zones of the reaction assembly with X′ different chemical composition of a fourth diversity introducing reaction step such that all reaction zones of the reaction assembly having a common x′ coordinate receive the same chemical composition of the fourth diversity introducing reaction step under conditions conducive to reaction of the solid support with the contacting chemical composition;
contacting the reaction zones of the reaction assembly with Y′ different chemical composition of a fifth diversity introducing reaction step such that all reaction zones of the reaction assembly having a common y′ coordinate receive the same chemical composition of the fifth diversity introducing reaction step under conditions conducive to reaction of the solid support with the contacting chemical composition such that a library of compounds is prepared where the solid support(s) of each reaction zone have a different reaction history and each compound of the library can be identified by five coordinates.

11. A method of claim 5 wherein each reaction zone is uniquely identifiable by four coordinates and a library of compounds is prepared by contacting each reaction zone of the library with a different combination of chemical compositions in at least four diversity introducing reaction steps.

12. A method of claim 11 for preparing a library of compounds comprising:
contacting a plurality of batches of solid supports with different chemical compositions of a first diversity introducing reaction step under conditions conducive for a reaction of the solid support with the chemical composition of the first diversity introducing reaction step such that there are Z batches of solid supports for Z different chemical compositions of the first diversity introducing reaction step and there are sufficient solid supports in each batch so that at least one solid support from a batch can be introduced into each reaction zone of the reaction assembly with a common z coordinate;
distributing solid supports to reaction zones of a reaction assembly such that reaction zones with a common z coordinate each receive a solid support from the same batch;
contacting the reaction zones of the reaction assembly with X different chemical compositions of a second diversity introducing reaction step such that all reaction zones of the reaction assembly having a common x coordinate receive the same chemical composition of the second diversity introducing reaction step under conditions conducive to reaction of the solid support with the contacting chemical composition;
contacting the reaction zones of the reaction assembly with Y different chemical compositions of a third diversity introducing reaction step such that all reaction zones of the reaction assembly having a common y coordinate receive the same chemical composition of a third diversity introducing reaction step under conditions conducive to reaction of the solid support with the contacting chemical composition;
contacting the reaction zones of the reaction assembly with X′ different chemical composition of a fourth diversity introducing reaction step such that all reaction zones of the reaction assembly having a common x′ coordinate receive the same chemical composition of the fourth diversity introducing reaction step under conditions conducive to reaction of the solid support with the contacting chemical composition such that a library of compounds is prepared where the solid support(s) of each reaction zone have a different reaction history and each compound of the library can be identified by four coordinates.

13. A method of claim 5, wherein the reaction assembly comprises a plurality of reaction columns.

14. A method of claim 13 wherein each reaction column comprises two or more reaction zones.

15. A method of claim 14 wherein solid supports contained in adjacent reaction zones of a reaction column are in physical contact and unable to exchange positions in the reaction column.

16. A method of claim 5 further comprising:
f) contacting each solid support with a chemical composition in a cleavage reaction such that a compound with a synthetic history corresponding to the coordinates of the reaction zone is cleaved from the support contained in the reaction zone.

17. A method of claim 5 where the chemical composition contacted with each solid support in the last diversity introducing reaction step is also the chemical composition for cleaving from the solid support such that a compound with a synthetic history corresponding to the coordinates of the reaction zone is cleaved from the support contained in the reaction zone.

18. A method for preparing a spatially separated library of compounds, comprising:
providing a reaction assembly comprising a plurality of reaction zones where each reaction zone can be defined by four or more coordinates and at least one set of two coordinates define a composite coordinate parameter;
introducing at least one solid support into each reaction zone; and
contacting each solid support with four or more diversity introducing reaction steps.

19. A reaction assembly for preparing a spatially separated library of compounds wherein the reaction assembly comprises:
at least one reaction block;
a plurality of reaction zones disposed in the reaction block(s) of the reaction assembly wherein each reaction zone of the reaction assembly is uniquely addressable with four or more coordinates; and
a plurality of solid supports located in the plurality of reaction zones where each solid support comprises at least one functional group that can react with a linker compound or a building block component of the combinatorial library such that at least one molecule of a composition corresponding to a compound of the combinatorial library of compounds or a compound that is a precursor to a compound of the combinatorial library of compounds can be bound to the solid support.

20. A reaction assembly of claim 19, wherein the reaction assembly comprises a plurality of reaction blocks arranged in a three dimensional array of reaction blocks having at least three coordinates to define each reaction block of the reaction assembly and at least six coordinates to define each reaction zone of the reaction assembly.

21. A reaction assembly of claim 19, wherein the reaction assembly comprises a plurality of reaction blocks arranged in a two-dimensional array of reaction blocks having at least two coordinates to define each reaction block of the reaction assembly and at least five coordinates to define each reaction zone of the reaction assembly.

22. A reaction assembly of claim 19, wherein the reaction assembly comprises a plurality of reaction blocks arranged in a one-dimensional array of reaction blocks having at least one coordinate to define each reaction block of the reaction assembly and at least four coordinates to define each reaction zone of the reaction assembly.

23. A reaction assembly of claim 19, for preparing a spatially separated library of compounds comprising:
a three-dimensional array of reaction blocks having x′, y′ and z′ axes and defining (x′, y′), (x′, z′) and (y′, z′) reaction block planes where each reaction block plane defines a set of reaction blocks that have a common coordinate, e.g., an (x′, y′) reaction block plane comprises reaction blocks of the reaction assembly that have a common z′ coordinate but different (x′, y′) coordinates;
each reaction block comprises a plurality of reaction zones arranged in three-dimensional array having x, y and z axes and defining (x, y), (x, z) and (y, z) reaction planes where each reaction plane defines a set of reaction zones that have a common coordinate, e.g., an (x, y) reaction plane comprises reaction zones of the reaction block that have a common z coordinate but different (x, y) coordinates; and
each reaction zone of the reaction assembly is uniquely identifiable by six coordinates, e.g., (x, y, z, x′, y′, z′) which define the location of the reaction zone in a reaction block and the position of the reaction block that comprises the specified reaction zone in the reaction assembly.

24. A reaction assembly of claim 19, for preparing a spatially separated library of compounds comprising:
a two-dimensional array of reaction blocks having x′ and y′ axes;
each reaction block comprises a plurality of reaction zones arranged in three-dimensional array having x, y and z axes and defining (x, y), (x, z) and (y, z) reaction planes where each reaction plane defines a set of reaction zones that have a common coordinate, e.g., an (x, y) reaction plane comprises reaction zones of the reaction block that have a common z coordinate but different (x, y) coordinates; and
each reaction zone of the reaction assembly is uniquely identifiable by five coordinates, e.g., (x, y, z, x′, y′) which define the location of the reaction zone in a reaction block and the position of the reaction block that comprises the specified reaction zone in the reaction assembly.

25. A reaction assembly of claim 19, for preparing a spatially separated library of compounds comprising:
an array of reaction blocks having an x′ axis;
each reaction block comprises a plurality of reaction zones arranged in three-dimensional array having x, y and z axes and defining (x, y), (x, z) and (y, z) reaction planes where each reaction plane defines a set of reaction zones that have a common coordinate, e.g., an (x, y) reaction plane comprises reaction zones of the reaction block that have a common z coordinate but different (x, y) coordinates; and
each reaction zone of the reaction assembly is uniquely identifiable by four coordinates, e.g., (x, y, z, x′) which define the location of the reaction zone in a reaction block and the position of the reaction block that comprises the specified reaction zone in the reaction assembly.

26. A reaction assembly of claim 19, wherein the reaction assembly comprises a single reaction block having at least one composite coordinate parameter such that each reaction zone of the single reaction block is addressable by four or more coordinates.

27. A reaction assembly of any one of claims 19 through 25, wherein each reaction block of the reaction assembly comprises a plurality of two-dimensional arrays of reaction zones.

28. The reaction assembly of claim 27, wherein the reaction zones of a reaction plane have a common z coordinate but different x and y coordinates.

29. A reaction assembly of any one of claims 19 through 26, for preparing a spatially separated library of compounds, the reaction assembly comprising:
reaction zones of a reaction block are arranged in a two-dimensional array of reaction columns where the reaction zones of the reaction block with common (x, y) coordinates are contained in a reaction column and a (x, y) reaction plane comprises the one reaction zone from each reaction column that have a common z coordinate.

30. A reaction assembly of claim 29, wherein solid supports contained in adjacent reaction zones of a reaction column are in physical contact and unable to exchange positions in the reaction column.

31. A reaction assembly for preparing a spatially separated library of compounds with four or more diversity introducing reaction steps comprising at least one composite reaction block comprising a plurality of reaction zones arrayed according to a Cartesian coordinate system defined by composite coordinate axes a, b, c wherein:
a=X(x′−1)+x;
b=Y(y′−1)+y;
c=Z(z′−1)+z;
X is the number of different chemical compositions for the diversity introducing chemical step that are contacted with reaction zones x coordinate values;
Y is the number of different chemical compositions for the diversity introducing chemical step that are contacted with reaction zones y coordinate values;
Z is the number of different chemical compositions for the diversity introducing chemical step that are contacted with reaction zones z coordinate values; and
x, y, z, x′, y′, z′ are six coordinates that define the six dimensions of a six dimensional library.
